(19) United States
(12) Patent Application Publication
(10) Pub. No.: US 2026/0259227 A1
(43) Pub. Date: Sep. 3, 2026

(54) PROTEIN BIOMARKERS FOR LANADELUMAB TREATMENT

(71) Applicant: Takeda Pharmaceutical Company Limited, Osaka-shi, Osaka (JP)

(72) Inventors: Daniel J. Sexton, Melrose, MA (US); Bin Li, Belmont, MA (US); Sin Kong Yeung, Hudson, MA (US)

(73) Assignee: Takeda Pharmaceutical Company Limited, Osaka-shi, Osaka (JP)

(21) Appl. No.: 18/877,564

(22) PCT Filed: Jun. 30, 2023
(86) PCT No.: PCT/IB2023/000402

§ 371 Date: Dec. 20, 2024

Related U.S. Application Data
(60) Provisional application No. 63/357,596, filed on Jun. 30, 2022.
(60) Provisional application No. 63/458,740, filed on Apr. 12, 2023.

(51) Int. Cl. G01N 33/68

(52) U.S. Cl. CPC G01N 33/6893; G01N 2800/22; G01N 2800/52

(57) ABSTRACT

Provided herein are methods for determining whether a disorder is susceptible to treatment with a plasma kallikrein inhibitor and methods for identifying a subject as a candidate for treatment with a plasma kallikrein inhibitor. Provided herein are methods of identifying a subject as having or being at risk for a disorder and methods of treating a disorder in a subject. Also provided herein are methods of evaluating effectiveness of a treatment in a subject.

### RELATED APPLICATIONS

The application claims the benefit under 35 U.S.C. 119 (e) of U.S. Provisional Application No. 63/357,596 filed on Jun. 30, 2022, and U.S. Provisional Application No. 63/458,740 filed on Apr. 12, 2023, each of which is incorporated by reference in its entirety.

### REFERENCE TO AN ELECTRONIC SEQUENCE LISTING

The contents of the electronic sequence listing (D061770139WO00-SEQ-JRV.xml; Size: 13,585 bytes; and Date of Creation: Jun. 29, 2023) is herein incorporated by reference in its entirety.

### BACKGROUND

Lanadelumab (TAKHZYRO®/Takeda) is a human monoclonal antibody that inhibits active plasma kallikrein and has been approved by several regulatory authorities for the treatment of hereditary angioedema (HAE) in human patients. HAE is a disease that causes episodic attacks of swelling, which can affect multiple parts of the body such as the face, extremities, genitals, GI tract, and upper airways. Because HAE symptoms often resemble symptoms of allergies or intestinal colics, HAE attacks are often difficult to identify until patients exhibit severe or life-threatening symptoms. Early diagnosis would allow for better management of emergency situations involving acute HAE attacks and would also help manage HAE patients to prevent or dampen acute HAE episodes, e.g., by allowing an HAE sufferer to avoid exposure to stimuli that might trigger HAE episodes.

### SUMMARY OF THE PRESENT DISCLOSURE

The plasma contact activation system is a pro-inflammatory and pro-coagulant system involving a group of plasma proteases. It is activated by either active Factor XII (FXIIa) upon exposure to foreign or negatively charged surfaces or on endothelial cell surfaces by prolylcarboxypeptidases (Sainz I. M. et al., Thromb. Haemost. (2007) 98, 77-83). Inappropriate or unregulated activation of the contact system has been implicated in various diseases, including hereditary angioedema (HAE). It is therefore of great interest to identify biomarkers for HAE and other diseases associated with the contact activation system in order to develop reliable diagnostic and prognostic methods for identifying subjects having or at risk of having HAE. Such biomarkers would also benefit studies to understand the underlying disease mechanisms, which could facilitate the development of effective new therapies.

The instant disclosure is based, at least in part, on the identification of protein biomarkers that are differentially present in biological samples obtained from subjects having HAE as compared to healthy individuals or differentially present in biological samples obtained from subjects in following treatment with a plasma kallikrein (pKal) inhibitor (i.e., lanadelumab), including protein biomarkers that become normalized (return to control levels) following treatment. The protein biomarkers identified herein, may be used, for example, in determining whether a disorder is susceptible to a treatment with a pKal inhibitor (e.g., lanadelumab), identifying diseases associated with the contact activation system, identifying patients who have or are at risk of a disorder, identifying a subject as a candidate for treatment, monitoring disease progression or disease state, and/or evaluating the efficacy of a treatment for the disorder.

Accordingly, aspects of the present disclosure provide methods for determining if a disorder is susceptible to treatment with a pKal inhibitor, comprising measuring the level of a biomarker set, which comprises at least one protein selected from Table 1, in a biological sample of a subject having the disorder, and identifying the disorder as being susceptible to treatment with a pKal inhibitor if the level of the biomarker set deviates from a reference value. In some embodiments, the method further comprises administering the pKal inhibitor to the subject if the disorder is identified as susceptible to treatment with the pKal inhibitor.

Aspects of the present disclosure provide methods for identifying a subject as a candidate for treatment with a pKal inhibitor, comprising providing a biological sample from the subject having, suspected of having, or at risk of having a disorder, and measuring the level of a biomarker set, which comprises at least one protein selected from Table 1, in the biological sample, wherein if the level of the biomarker set in the biological sample deviates from a reference value, the subject is identified as a candidate for treatment with the pKal inhibitor. In some embodiments, the method further comprises administering the pKal inhibitor to the subject identified as a candidate for treatment.

Aspects of the present disclosure provide methods for identifying a subject as having or at risk of having a disorder, comprising providing a biological sample from the subject, and measuring the level of a biomarker set, which comprises at least one protein selected from Table 1, in the biological sample, wherein if the level of the biomarker set in the biological sample deviates from the level of the biomarker set in a control sample, the subject is identified as having or at risk of the disorder. In some embodiments, the method further comprises administering to the subject an effective amount of a pKal inhibitor, if the subject is identified as having or at risk of having the disorder.

Aspects of the present disclosure provide methods for treating a disorder in a subject, comprising administering to the subject an effective amount of a plasma kallikrein (pKal) inhibitor, wherein the subject has a level of a biomarker set that deviates from the level of the biomarker set in a control sample, and wherein the biomarker set comprises at least one protein selected from Table 1. In some embodiments, the disorder is not hereditary angioedema (HAE).

Aspects of the present disclosure provide compositions for use in treating a subject having a disorder, the composition comprising a plasma kallikrein (pKal) inhibitor, wherein the subject has a level of a biomarker set that deviates from the level of the biomarker set in a control sample, wherein the biomarker set comprises at least one protein selected from Table 1.

In some embodiments, the pKal inhibitor is lanadelumab.

In some embodiments, the biomarker set consists of 2-10 proteins selected from Table 1. In some embodiments, the biological sample is a serum sample or a plasma sample.

In some embodiments, the disorder is a disease associated with the contact activation system. In some embodiments, the disorder is hereditary angioedema (HAE). In some embodiments, the disorder is type I HAE or type II HAE. In some embodiments, the disorder is not hereditary angioedema (HAE).

In some embodiments, the at least one protein is a kallikrein-kinin system protein selected from the group consisting of kallikrein-13 (KLK13), kallikrein-14 (KLK14), 2-chain high molecular weight kininogen (KNG1), and kininostatin. In some embodiments, the at least one protein is a blood coagulation protein selected from the group consisting of alpha-2-macroglobulin (A2M), complement C1q and tumor necrosis factor-related protein 9A (C1QTNF9), thrombin, and plasma serine protease inhibitor (SERPINA5). In some embodiments, the at least one protein is a cell adhesion protein selected from the group consisting of cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), and protocadherin gamma-C3 (PCDHGC3). In some embodiments, the at least one protein is a proteolysis-related protein selected from the group consisting of proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), and E3 ubiquitin-protein ligase ZNRF3 (ZNRF3). In some embodiments, the at least one protein is a complement activation protein selected from the group consisting of complement C1q and tumor necrosis factor-related protein 9A (C1QTNF9).

In some embodiments, the at least one protein is selected from the group consisting of thrombin (F2), tissue kallikrein 14 (KLK4), tissue kallikrein 13 (KLK13), Inter-α-trypsin inhibitor heavy chain H4 (ITIH4), α-macroglobulin, Apolipoprotein B (APOB), interleukin-21 (IL-21), complement component C3 (C3), kininogen (KNG1), Protein arginine N-methyltransferase 1 (PRMT1), and Complement C1q and tumor necrosis factor-related protein 9A (C1QTNF9).

In some embodiments, the biological sample is provided in an evacuated blood collection tube, which comprises one or more protease inhibitors. In some embodiments, the level of the biomarker set is measured by enzyme-linked immunosorbent assay (ELISA), an immunoblotting assay, or a lateral flow assay.

In some embodiments, the subject is a human patient.

Aspects of the present disclosure provide methods for evaluating a treatment in a subject, comprising measuring the level of a biomarker set, which comprises at least one protein selected from Table 1, in biological samples obtained from the subject before and after the treatment or during the course of the treatment, and evaluating effectiveness of the treatment based on the level of the biomarker set, wherein a deviation level of the biomarker set after the treatment or over the course of the treatment as compared to before the treatment, indicates that the treatment is effective on the subject.

In some embodiments, the treatment comprises administration of a plasma kallikrein (pKal) inhibitor to the subject. In some embodiments, the pKal inhibitor is lanadelumab.

In some embodiments, the biomarker set consists of 2-10 proteins selected from Table 1. In some embodiments, the biological samples are serum samples or a plasma samples.

In some embodiments, at least one protein is a kallikrein-kinin system protein selected from the group consisting of kallikrein-13 (KLK13), kallikrein-14 (KLK14), 2-chain high molecular weight kininogen (KNG1), and kininostatin. In some embodiments, at least one protein is a blood coagulation protein selected from the group consisting of alpha-2-macroglobulin (A2M), complement C1q and tumor necrosis factor-related protein 9A (C1QTNF9), thrombin, and plasma serine protease inhibitor (SERPINA5). In some embodiments, at least one protein is a cell adhesion protein selected from the group consisting of cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), and protocadherin gamma-C3 (PCDHGC3). In some embodiments, at least one protein is a proteolysis-related protein selected from the group consisting of proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), and E3 ubiquitin-protein ligase ZNRF3 (ZNRF3). In some embodiments, at least one protein is a complement activation protein selected from the group consisting of complement C1q and tumor necrosis factor-related protein 9A (C1QTNF9).

In some embodiments, the at least one protein is selected from the group consisting of thrombin (F2), tissue kallikrein 14 (KLK4), tissue kallikrein 13 (KLK13), Inter-α-trypsin inhibitor heavy chain H4 (ITIH4), α-macroglobulin, Apolipoprotein B (APOB), interleukin-21 (IL-21), complement component C3 (C3), kininogen (KNG1), Protein arginine N-methyltransferase 1 (PRMT1), and Complement C1q and tumor necrosis factor-related protein 9A (C1QTNF9).

In some embodiments, the biological samples are obtained in evacuated blood collection tubes, which comprise one or more protease inhibitors. In some embodiments, the level of the biomarker set is measured by enzyme-linked immunosorbent assay (ELISA), an immunoblotting assay, or a lateral flow assay.

In some embodiments, the subject is a human patient. In some embodiments, the subject has, is suspected of having, or is at risk for a disease associated with the contact activation system. In some embodiments, the disease associated with the contact activation system is hereditary angioedema (HAE). In some embodiments, the HAE is type I HAE or type II HAE. In some embodiments, the disease associated with the contact activation system is not HAE.

Aspects of the present disclosure provide methods of analyzing a sample comprising (i) providing a biological sample (e.g., serum sample or a plasma sample) obtained from a subject, such as a human subject, having, suspected of having, or being at risk for a disease associated with the contact activation system; and (ii) measuring the level of a biomarker set, which comprises at least one protein selected from Table 1, wherein if the biomarker set consists of one protein, said protein is not 2-chain high molecular weight kininogen (KNG1), alpha-2-macroglobulin (A2M), complement C1q and tumor necrosis factor-related protein 9A (C1QTNF9), thrombin, or interleukin-21 (IL-21).

In some embodiments, the biomarker set consists of 2-10 proteins selected from Table 1. In some embodiments, the biological sample is a serum sample or a plasma sample. In some embodiments, the disease associated with the contact activation system is hereditary angioedema (HAE). In some embodiments, the HAE is type I HAE or type II HAE.

In some embodiments, at least one protein of the biomarker set is a kallikrein-kinin system protein selected from the group consisting of kallikrein-13 (KLK13), kallikrein-14 (KLK14), 2-chain high molecular weight kininogen (KNG1), and kininostatin. In some embodiments, at least one protein of the biomarker set is a coagulation protein selected from the group consisting of alpha-2-macroglobulin (A2M), complement C1q and tumor necrosis factor-related protein 9A (C1QTNF9), thrombin, and plasma serine protease inhibitor (SERPINA5). In some embodiments, at least one protein of the biomarker set is a cell adhesion protein selected from the group consisting of cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), and protocadherin gamma-C3 (PCDHGC3). In some embodiments, at least one protein of the biomarker set is a proteolysis-related protein selected from the group consisting of proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), and E3 ubiquitin-protein ligase ZNRF3 (ZNRF3). In some embodiments, at least one protein of the biomarker set is a complement activation protein selected from the group consisting of complement C1q and tumor necrosis factor-related protein 9A (C1QTNF9).

In some embodiments, the at least one protein is selected from the group consisting of thrombin (F2), tissue kallikrein 14 (KLK4), tissue kallikrein 13 (KLK13), Inter-α-trypsin inhibitor heavy chain H4 (ITIH4), α-macroglobulin, Apolipoprotein B (APOB), interleukin-21 (IL-21), complement component C3 (C3), kininogen (KNG1), Protein arginine N-methyltransferase 1 (PRMT1), and Complement C1q and tumor necrosis factor-related protein 9A (C1QTNF9).

In some embodiments, providing a biological sample comprises collecting the biological sample into an evacuated blood collection tube, which comprises one or more protease inhibitors. In some embodiments, the measuring the level of a biomarker set is performed using an enzyme-linked immunosorbent assay (ELISA), an immunoblotting assay, or a lateral flow assay.

In some embodiments, the subject is a human patient. In some embodiments, the method further comprises identifying the subject as having a disease associated with the contact system, if the level of the biomarker set of the subject deviates from the level of the same biomarker set of a control subject. In some embodiments, the method further comprises administering to the subject an effective amount of a therapeutic agent for treating the disease, if the subject is identified as having the disease.

In some embodiments, the subject is a human patient who is on a treatment for the disease. In some embodiments, the method further comprises assessing the efficacy of the treatment by comparing the level of the biomarker set measured in a biological sample obtained from the subject after treatment or during the course of treatment to the level of the same biomarker set measured in a biological sample obtained from the subject before treatment, wherein if the level of the biomarker set in the sample obtained after treatment or during the course of treatment deviates from the level of the biomarker set in the sample obtained before treatment, then the treatment is determined to be effective. In some embodiments, the method further comprises assessing the efficacy of the treatment by comparing the level of the biomarker set measured in a biological sample obtained from the subject after treatment or during the course of treatment to the level of the same biomarker set measured in a control sample obtained from a healthy subject, wherein if the level of the biomarker set in the sample obtained after treatment or during the course of treatment does not deviate from the level of the biomarker set in the control sample, then the treatment is determined to be effective. In some embodiments, the method further comprises administering to the subject an effective amount a therapeutic agent for treating the disease if the treatment is not determined to be effective. In some embodiments, the subject is administered an increased dose of the therapeutic agent if the subject has been administered the therapeutic agent previously as part of the course of treatment.

In some embodiments, a therapeutic agent administered to the subject is a plasma kallikrein (pKal) inhibitor, a bradykinin 2 receptor inhibitor, and/or a C1 esterase inhibitor. In some embodiments the pKal inhibitor is an anti-pKal antibody (e.g., lanadelumab) or an inhibitory peptide (e.g., ecallantide). In some embodiments, a bradykinin 2 receptor inhibitor is an inhibitory peptide (e.g., icatibant). In some embodiments, a C1 esterase inhibitor is a human plasma-derived C1 esterase inhibitor.

Aspects of the present disclosure provide kits for analyzing a sample of a subject having, suspected of having, or at risk for a disease associated with the contact system, the kit comprising a first binding agent specific to a first protein biomarker selected from Table 1; and a second binding agent specific to a second protein biomarker selected from Table 1; wherein the first protein biomarker and the second protein biomarker are different. In some examples, the first and/or the second binding agent is an antibody specific to the protein marker. In some embodiments, the kit may further comprise a first detection agent that binds to the first binding agent and a second detection agent that binds to the second binding agent. In some embodiments, the first binding agent and the second binding agent are immobilized on a support member.

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows a volcano plot of plasma proteins analyzed via a multiplexed approach using DNA aptamers specific for protein analytes (SomaScan®). The plot presents comparisons of analyte levels in citrated plasma from hereditary angioedema (HAE) patients (baseline) as compared to citrated plasma from healthy subjects (controls). Proteins previously known to be differentially present in plasma of HAE patients are labeled. Statistical significance is indicated by the dashed line (p>0.05).

FIG. 2 shows a plot of expression analysis of C1 esterase inhibitor (C1-INH) in plasma samples from healthy controls and HAE patients at baseline, as determined by SomaScan® aptamer binding.

FIG. 3 shows a plot of expression analysis of complement component C4 in plasma samples from healthy controls and HAE patients at baseline, as determined by SomaScan® aptamer binding.

FIGS. 4A and 4B show plots of expression analysis of cleaved HMWK (cHMWK) in plasma of healthy controls, HAE patients at baseline, and HAE patients after 26 weeks of lanadelumab treatment, as determined by SomaScan® aptamer binding. FIG. 4A shows expression comparisons between healthy controls, HAE patients at baseline, and HAE patients after 26 weeks of lanadelumab treatment. FIG. 4B shows cHMWK expression in patient-matched samples before treatment (baseline) and post-treatment (Week26).

FIG. 5 shows a plot of expression analysis of F2 (thrombin) in plasma samples of healthy controls, HAE patients at baseline, and HAE patients after 26 weeks of lanadelumab treatment (Week26), as determined by SomaScan® aptamer binding.

FIG. 6 shows a plot of expression analysis of inter-α-trypsin inhibitor heavy chain H4 (ITH4) in plasma samples of healthy controls, HAE patients at baseline, and HAE patients after 26 weeks of lanadelumab treatment (Week26), as determined by SomaScan® aptamer binding.

FIG. 7 show a plot of expression analysis of interleukin-36A (IL-36A) in plasma samples of healthy controls, HAE patients at baseline, and HAE patients after 26 weeks of lanadelumab treatment (Week26), as determined by SomaScan® aptamer binding.

FIG. 8 shows a plot of expression analysis of interleukin-21 (IL-21) in plasma samples of healthy controls, HAE patients at baseline, and HAE patients after 26 weeks of lanadelumab treatment (Week26), as determined by SomaScan® aptamer binding.

FIGS. 9A-9G show plots of expression analysis of protein biomarkers determined by SomaScan® aptamer binding. Protein levels are shown for plasma samples of healthy controls, HAE patients at baseline, and HAE patients after 26 weeks of lanadelumab treatment (Week26). FIG. 9A shows levels of apolipoprotein B (APOB). FIG. 9B shows levels of kininostatin (KNG1). FIG. 9C shows levels of G antigen 2 (GAGE2B). FIG. 9D shows levels of mortality factor 4-like protein 2 (MORF4L2). FIG. 9E shows levels of complement C1q and tumor necrosis factor-related protein 9A (C1QTNF9). FIG. 9F shows levels of plasma serine protease inhibitor, serpin family A member 5 (SERPINA5). FIG. 9G shows levels of cadherin-15:cytoplasmic domain (CDH15).

FIGS. 10A-10F show plots of expression analysis of protein biomarkers determined by SomaScan® aptamer binding. Protein levels are shown for plasma samples of healthy controls, HAE patients at baseline, and HAE patients after 26 weeks of lanadelumab treatment (Week26). FIG. 10A shows levels of alpha-2-macroglobulin (A2M). FIG. 10B shows levels of Interleukin-12 (IL-12A/IL-12B). FIG. 10C shows levels of liver-expressed antimicrobial peptide 2 (LEAP2). FIG. 10D shows levels of kininogen (KNG1). FIG. 10E shows levels of tissue kallikrein-13 (KLK13). FIG. 10F shows levels of tissue kallikrein-14 (KLK14).

FIG. 11 is a schematic of a local network analysis using a causal-associational network (CASNET) approach, which identified 120 disease state biomarkers that are impacted by treatment with lanadelumab.

FIG. 12 is a schematic of a local network analysis using a causal-associational network (CASNET) approach, incorporated into a known knowledge network. Proteins that were found to be increased in plasma samples of HAE patients at baseline as compared to healthy controls are indicated with “+” and include thrombin (F2), apolipoprotein D (APOD), α-macroglobulin (A2M), apolipoprotein B (APOB), complement component C3 (C3), and kininogen (KNG1). Proteins that were found to be decreased in plasma samples of HAE patients at baseline as compared to healthy controls are indicated with “*” and include protein arginine N-methyltransferase 1 (PRMT1), inter-α-trypsin inhibitor heavy chain H4 (ITIH4), tissue kallikrein 14 (KLK14), tissue kallikrein 13 (KLK13), complement C1q and tumor necrosis factor-related protein 9A (C1QTNF9), and GTP-binding protein SAR1b (SAR1B). Other proteins were incorporated based on pathway associations.

FIGS. 13A-13H show plots of expression analysis of Kininogen (KNG1) in plasma of healthy controls, HAE patients at baseline, and HAE patients after 26 weeks of lanadelumab treatment, as determined by SomaScan® aptamer binding. FIG. 13A shows KNG1 (ID 15343-337, Table 1) expression comparisons between healthy controls, HAE patients at baseline, and HAE patients after 26 weeks of lanadelumab treatment. FIG. 13B shows KNG1 (ID 15343-337, Table 1) expression in patient-matched samples before treatment (baseline) and post-treatment (Week26). FIG. 13C shows KNG1 (ID 19631-13, Table 1) expression comparisons between healthy controls, HAE patients at baseline, and HAE patients after 26 weeks of lanadelumab treatment. FIG. 13D shows KNG1 (ID 19631-13, Table 1) expression in patient-matched samples before treatment (baseline) and post-treatment (Week26). FIG. 13E shows KNG1 (ID 4918-21, Table 1) expression comparisons between healthy controls, HAE patients at baseline, and HAE patients after 26 weeks of lanadelumab treatment. FIG. 13F shows KNG1 (ID 4918-21, Table 1) expression in patient-matched samples before treatment (baseline) and post-treatment (Week26). FIG. 13G shows KNG1 (ID 7784-1, Table 1) expression comparisons between healthy controls, HAE patients at baseline, and HAE patients after 26 weeks of lanadelumab treatment. FIG. 13H shows KNG1 (ID 7784-1, Table 1) expression in patient-matched samples before treatment (baseline) and post-treatment (Week26).

FIGS. 14A-14D shows plots of expression analyses of Complement C3 (C3) in plasma of healthy controls, HAE-C1-INH patients at baseline, and HAE-C1-INH patients after 26 weeks of lanadelumab treatment, as determined by SomaScan® aptamer binding. FIG. 14A shows C3 (ID 2754-50, Table 1) expression comparisons between healthy controls, HAE-C1-INH patients at baseline, and HAE-C1-INH patients after 26 weeks of lanadelumab treatment. FIG. 14B shows C3b (ID 4480-59, Table 1) expression comparisons between healthy controls, HAE-C1-INH patients at baseline, and HAE-C1-INH patients after 26 weeks of lanadelumab treatment. FIG. 14C shows C3b, inactivated (ID 2683-1, Table 1) expression comparisons between healthy controls, HAE-C1-INH patients at baseline, and HAE-C1-INH patients after 26 weeks of lanadelumab treatment. FIG. 14D shows C3d (ID 5803-24, Table 1) expression comparisons between healthy controls, HAE-C1-INH patients at baseline, and HAE-C1-INH patients after 26 weeks of lanadelumab treatment.

FIGS. 15A-15C show plots of protein analyses of protein biomarkers determined by ELISA in plasma samples of HAE-C1-INH subjects treated every 4 weeks with 150 mg or 300 mg of lanadelumab and plasma samples of healthy controls. The plasma samples from HAE-C1-INH subjects were collected at baseline (pre-dose; day 0) and after 98 days of lanadelumab treatment. FIG. 15A shows protein levels of alpha-2-macroglobulin (A2M). FIG. 15B shows protein levels of Apolipoprotein B (APOB). FIG. 15C shows protein levels of interleukin-21 (IL-21).

### DETAILED DESCRIPTION

The contact activation system initiates the intrinsic pathway of coagulation and promotes inflammation through the release of the proinflammatory peptide bradykinin. Factor XII (FXII), also known as Hageman Factor, is a serine protease that plays a role in activation of the intrinsic pathways of coagulation as well as the kallikrein-kinin system. FXII is activated by negatively charged surfaces (e.g., polyanionic surfaces, glass, polyphosphate, ellagic acid) to produce the active form FXIIa. Activated FXIIa has the ability to cleave pre-kallikrein, generating active pKal. Subsequently, activated pKal is able to cleave FXII into FXIIa, resulting in a positive feedback loop in which FXIIa generates even more pKal, which further activates additional FXII into FXIIa. Activated pKal is also able to cleave high molecular weight kininogen (HMWK) to release bradykinin. In diseases associated with contact system activation, such as HAE, increased levels of bradykinin can induce vasodilation and inflammation that result in edematous HAE attacks. It is desired to identify novel biomarkers that can be used, for example, to identify diseases as mediated by the contact activation system, identify subjects having or being at risk of having such a disease, as well as identify additional diseases that may benefit from treatment with a plasma kallikrein inhibitor (e.g., lanadelumab/TAKHZYRO®).

The present disclosure is based, at least in part, on the identification of proteins that are differentially present in biological samples obtained from subjects having HAE as compared to healthy individuals via proteomic analysis. It was observed that a number of proteins were found to be present in subjects having HAE that deviated from the levels in healthy (control) subjects. Interestingly, it was unexpectedly observed that many proteins normalized (returned to control levels) following treatment with lanadelumab, a pKal inhibitor. Groups of proteins predicted to belong to particular cellular pathways or processes (e.g., proteins involved in mitochondrial function, proteolysis, blood coagulation, etc.) and proteins belonging to protein families (e.g., interleukins) were found to have similar trends (e.g., elevated or reduced levels) in samples from subjects having the disease as compared to healthy individuals.

Accordingly, provided herein are methods for determining if a disorder is susceptible to treatment with a plasma kallikrein (pKal) inhibitor and methods of identifying a subject as a candidate for treatment with a pKal inhibitor. Also provided herein are methods for analyzing biological samples from subjects having, suspected of having, or being at risk for a disease or disorder in which one or more of the protein biomarkers deviates from control levels, based on detecting the presence or measuring the level of a protein biomarker or protein biomarker set. Such methods may be useful, e.g., for identifying patients who are at risk of a disease, selecting a candidate for treatment, monitoring disease progression or disease state, assessing the efficacy of a treatment against a disease, determining a course of treatment, assessing whether a subject is at risk for an attack of the disease, and/or for research purposes, including, e.g., studying the mechanism of a disease and/or biological pathways/processes involved in the disease, which may be relied upon for the development of new therapies.

### Protein Biomarkers

The methods and kits described herein are based, at least in part, on the identification of proteins that were found to be differentially present in samples from subjects having HAE as compared with samples from healthy subjects, and/or differentially present in samples from subjects at different stages of such a disease (e.g., at baseline as compared to following treatment with lanadelumab), including proteins that were found to be differentially present in samples from subjects having HAE as compared with samples from healthy subjects, which returned (normalized) to the level of healthy subjects following treatment with lanadelumab. As used herein, the term “protein biomarker” or “protein biomarker set” refers to a protein or set of proteins that are present at different levels in samples from different groups of subjects, for example, subjects having a disease versus healthy subjects (e.g., subjects who are free of the disease), or subjects having the disease and being at the quiescence stage versus subjects experiencing an acute stage of the disease. In some embodiments, a protein or set of proteins that are present at different levels in samples from different groups of subjects, such as subjects having a disease (e.g., at baseline, prior to treatment) versus subjects that have been administered one or more treatments for the disease.

Such biomarker/biomarker sets may be used in both clinical applications and non-clinical applications (for example, for research purposes).

In some embodiments, a protein biomarker may be present at an elevated level in samples from subjects having a disease as compared to the level of the same protein biomarker in samples from healthy subjects. In some embodiments, a protein biomarker may be present at a reduced level in samples from subjects having a disease as compared to the level of the biomarker in samples from healthy subjects. In yet other instances, a protein biomarker may be present at an elevated level in samples obtained from subjects experiencing an acute stage of a disease as described herein as compared with subjects during disease quiescence (baseline). Alternatively, a protein biomarker may be present at a reduced level in samples obtained from subjects experiencing an acute stage of a disease as described herein as compared with subjects during disease quiescence (baseline).

In some embodiments, a protein biomarker set containing one or more biomarkers can be analyzed in the methods described herein. When the protein biomarker set contains more than one biomarker, all of the biomarkers may present at elevated levels or reduced levels in subjects having a disease as compared with healthy subjects. Alternatively, a protein biomarker set may contain at least one biomarker that is elevated in subjects having the disease as compared with healthy subjects and at least one biomarker that is reduced in subjects having the disease as compared with healthy subjects.

Similarly, a protein biomarker set for evaluating whether a treatment is effective in treating a disorder in a subject, the biomarker set may contain multiple biomarkers that are all elevated or reduced in samples obtained at baseline (prior to treatment) as compared with levels in samples obtained following treatment. Alternatively, the biomarker set may contain at least one biomarker that is elevated in samples obtained at baseline (prior to treatment) and at least one biomarker that is reduced in the obtained at baseline (prior to treatment) as compared with levels in samples obtained following treatment.

Table 1 below provides biomarkers that can be evaluated by the methods described herein to evaluate subjects or biological samples from subjects, for example for identifying and/or treating subjects having or suspected of having, or being at risk for a disorder, determining if a disorder is susceptible for treatment with a plasma kallikrein inhibitor, and/or for evaluating the efficacy of a treatment of a disorder.

In some embodiments, the biomarker set to be measured and analyzed in any of the methods described herein includes at least one (e.g., 1, 2, 3, 4, 5, 6, 7, 8, 9, 10, or more) proteins selected from Table 1. In some embodiments, when the biomarker set includes a single protein, that protein is not 2-chain high molecular weight kininogen (HMWK; KNG1), alpha-2-macroglobulin (A2M), complement C1q and tumor necrosis factor-related protein 9A (C1QTNF9), thrombin, or interleukin-21 (IL-21). In some examples, the protein biomarker set to be measured and analyzed in a method described herein does not include a combination of any two or more of 2-chain high molecular weight kininogen (HMWK; KNG1), alpha-2-macroglobulin (A2M), complement C1q and tumor necrosis factor-related protein 9A (C1QTNF9), thrombin, and/or interleukin-21 (IL-21).

As described in Example 1, it was found that several proteins involved in the kallikrein-kinin system were differentially present in samples from subjects having HAE before treatment as compared to healthy subjects and/or were normalized (e.g., returned to levels comparable to healthy subjects) in samples from subjects having HAE after treatment as compared to healthy subjects. In some embodiments, the biomarker set includes one or more kallikrein-kinin system proteins as listed in Table 1. In some embodiments, the kallikrein-kinin system protein of the biomarker set includes kallikrein-13 (KLK13), kallikrein-14 (KLK14), 2-chain high molecular weight kininogen (KNG1), or kininostatin, or a combination thereof.

As described in Example 1, it was found that several proteins involved in the blood coagulation pathway were differentially present in samples from subjects having HAE before treatment as compared to healthy subjects and/or were normalized (e.g., returned to levels comparable to healthy subjects) in samples from subjects having HAE after treatment as compared to healthy subjects. In some embodiments, the biomarker set includes one or more blood coagulation proteins as listed in Table 1. In some embodiments, the blood coagulation protein of the biomarker set includes alpha-2-macroglobulin (A2M), complement C1q and tumor necrosis factor-related protein 9A (C1QTNF9), thrombin, or plasma serine protease inhibitor (SERPINA5), or a combination thereof.

As described in Example 1, it was found that several proteins involved in cell adhesion were differentially present in samples from subjects having HAE before treatment as compared to healthy subjects and/or were normalized (e.g., returned to levels comparable to healthy subjects) in samples from subjects having HAE after treatment as compared to healthy subjects. In some embodiments, the biomarker set includes one or more proteins involved in cell adhesion as listed in Table 1. In some embodiments, the protein involved in cell adhesion of the biomarker set includes cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), or protocadherin gamma-C3 (PCDHGC3), or a combination thereof.

In some embodiments, the biomarker set includes one or more proteins involved in cardiovascular function as listed in Table 1. In some embodiments, the protein involved in cardiovascular function of the biomarker set includes apolipoprotein B (APOB).

As described in Example 1, it was found that several proteins involved in proteolysis, including proteases and proteolytic inhibitors, were differentially present in samples from subjects having HAE before treatment as compared to healthy subjects and/or were normalized (e.g., returned to levels comparable to healthy subjects) in samples from subjects having HAE after treatment as compared to healthy subjects. In some embodiments, the biomarker set includes one or more proteins involved in proteolysis as listed in Table 1. In some embodiments, the proteolysis-related protein of the biomarker set includes proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), or E3 ubiquitin-protein ligase ZNRF3 (ZNRF3), or a combination thereof. In some embodiments, the proteolysis-related protein of the biomarker set includes thrombin (F2), tissue kallikrein 14 (KLK14), inter-α-trypsin inhibitor heavy chain H4 (ITIH4), or α-macroglobulin (A2M), or a combination thereof.

As described in Example 1, it was found that several proteins involved in complement activation were differentially present in samples from subjects having HAE before treatment as compared to healthy subjects and/or were normalized (e.g., returned to levels comparable to healthy subjects) in samples from subjects having HAE after treatment as compared to healthy subjects. In some embodiments, the biomarker set includes one or more proteins involved in complement activation as listed in Table 1. In some embodiments, the complement activation protein of the biomarker set is complement C1q and tumor necrosis factor-related protein 9A (C1QTNF9).

In some embodiments, the biomarker set includes one or more proteins that are cytokines as listed in Table 1. In some embodiments, the cytokine of the biomarker set includes interleukin-12 (IL-12A/IL-12B), interleukin 21 (IL-21), or interleukin 7 (IL-7), or a combination thereof.

TABLE 1
Contact System Activation Biomarkers

| | | | | Healthy | HAE | HAE |
|---|---|---|---|---|---|---|
| | | | | Control | Baseline | Week 26 |
| | | | | (Log2 | (Log2 | (Log2 |
| | | | | Fluorescence | Fluorescence | Fluorescence |
| Protein | Gene | ID | UniProt ID | units) | units) | units) |
| Alpha-2-macroglobulin | A2M | 3708-62 | P01023 | 14.05196 | 14.335554 | 14.027218 |
| Adenosine deaminase | ADA | 19751-21 | P00813 | 12.162506 | 12.011137 | 12.133948 |
| CD97 antigen | ADGRE5 | 2822-56 | P48960 | 12.038216 | 11.697353 | 11.848497 |
| Apolipoprotein B | APOB | 2797-56 | P04114 | 10.984104 | 11.625263 | 11.119179 |
| Apolipoprotein D | APOD | 8262-20 | P05090 | 12.220463 | 12.512348 | 12.254823 |
| Asparagine synthetase | ASNS | 23694-3 | P08243 | 11.801263 | 11.527334 | 11.669931 |
| BMP and activin | BAMBI | 6935-123 | Q13145 | 11.379663 | 11.166142 | 11.254385 |
| membrane-bound | | | | | | |
| inhibitor | | | | | | |
| homolog: Cytoplasmic | | | | | | |
| domain | | | | | | |
| Bcl-2-like protein 1 | BCL2L1 | 4423-77 | Q07817 | 11.133034 | 10.937268 | 11.062571 |
| Complement C1q and | C1QTNF9 | 6019-12 | P0C862 | 11.638914 | 11.41087 | 11.527181 |
| tumor necrosis factor- | | | | | | |
| related protein 9A | | | | | | |
| Complement C3 | C3 | 2754-50 | P01024 | 15.785446 | 16.517944 | 16.154523 |
| Complement C3b | C3b | 4480-59 | P01024 | 12.567962 | 15.076424 | 14.195009 |
| Complement C3b, | iC3b | 2683-1 | P01024 | 14.688529 | 16.025737 | 15.326262 |
| inactivated | | | | | | |
| Complement C3d | C3d | 5803-24 | P01024 | 12.677868 | 13.583916 | 13.110022 |
| fragment | | | | | | |
| Caspase-4 | CASP4 | 22092-43 | P49662 | 10.3271 | 10.158743 | 10.250013 |
| CMRF35-like molecule 7 | CD300LB | 10713-151 | A8K4G0 | 7.088381 | 7.224614 | 7.162122 |
| T-cell surface | CD3E | 18870-1 | P07766 | 9.988589 | 9.795452 | 9.920559 |
| glycoprotein CD3 epsilon | | | | | | |
| chain | | | | | | |
| CD6 | CD6 | 20519-7 | P30203 | 7.855053 | 7.967546 | 7.909131 |
| Cell division control | CDC42 | 9840-2 | P60953 | 10.847959 | 10.194991 | 10.615179 |
| protein 42 homolog | | | | | | |
| Cadherin-1 | CDH1 | 18429-10 | P12830 | 6.87047 | 7.245357 | 7.137691 |
| Cadherin-15: Cytoplasmic | CDH15 | 11215-6 | P55291 | 11.794256 | 11.381274 | 11.714125 |
| domain | | | | | | |
| Chromogranin-A | CHGA | 11184-51 | P10645 | 15.184195 | 14.573416 | 14.961358 |
| Choline/ethanolamine | CHKB | 13117-232 | Q9Y259 | 12.140662 | 12.643254 | 12.3626 |
| kinase | | | | | | |
| Defensin-5 | DEFA5 | 6387-61 | Q01523 | 12.528811 | 12.116836 | 12.297561 |
| Beta-defensin 132 | DEFB132 | 9022-49 | Q7Z7B7 | 9.93264 | 9.692778 | 9.832714 |
| Dynamin-1-like protein | DNM1L | 21452-3 | O00429 | 12.39159 | 12.000977 | 12.175476 |
| Dipeptidase 2 | DPEP2 | 8327-26 | Q9H4A9 | 13.461814 | 13.265539 | 13.383243 |
| Dual specificity tyrosine- | DYRK2 | 20205-55 | Q92630 | 7.875799 | 7.999642 | 7.939391 |
| phosphory lation-regulated | | | | | | |
| kinase 2 | | | | | | |
| EF-hand domain- | EFHD1 | 19616-100 | Q9BUP0 | 10.662215 | 10.781956 | 10.700262 |
| containing protein D1 | | | | | | |
| Eukaryotic translation | EIF1B | 12701-1 | O60739 | 11.603911 | 11.104707 | 11.447564 |
| initiation factor 1b | | | | | | |
| Ephrin type-A receptor 2 | EPHA2 | 4834-61 | P29317 | 10.120486 | 9.978342 | 10.024347 |
| Endoplasmic reticulum | ERP44 | 6064-4 | Q9BS26 | 12.679348 | 12.971754 | 12.861212 |
| resident protein 44 | | | | | | |
| S-formylglutathione | ESD | 4984-83 | P10768 | 9.312142 | 9.456262 | 9.385553 |
| hydrolase | | | | | | |
| Thrombin | F2 | 4157-2 | P00734 | 10.668153 | 11.413064 | 10.626529 |
| Uncharacterized protein | FAM241B | 8220-15 | Q96D05 | 13.227636 | 13.030458 | 13.151032 |
| C10orf35 | | | | | | |
| Glutamate | FOLH1 | 3218-8 | Q04609 | 12.442219 | 12.130291 | 12.25063 |
| carboxypeptidase 2 | | | | | | |
| G antigen 2 | GAGE2B | 25413-80 | Q13066 | 11.633228 | 11.992673 | 11.803497 |
| Galactosylceramide | GAL3ST1 | 8696-15 | Q99999 | 12.936022 | 12.639141 | 12.780522 |
| sulfotransferase | | | | | | |
| Guanylate-binding protein | GBP6 | 7818-101 | Q6ZN66 | 11.843824 | 11.670461 | 11.773943 |
| 6 | | | | | | |
| Gem-associated protein 6 | GEMIN6 | 21614-2 | Q8WXD5 | 9.726709 | 9.868998 | 9.789051 |
| Glutaredoxin-3 | GLRX3 | 16596-25 | O76003 | 11.940294 | 11.300856 | 11.635328 |
| Neuroendocrine secretory | GNAS | 5757-45 | O95467 | 9.218081 | 9.305605 | 9.280946 |
| protein 55 | | | | | | |
| Aspartate | GOT1 | 4912-17 | P17174 | 10.294335 | 10.446204 | 10.377776 |
| aminotransferase, | | | | | | |
| cytoplasmic | | | | | | |
| Iron-sulfur cluster co- | HSCB | 19316-2 | Q8IWL3 | 11.513623 | 11.364342 | 11.463744 |
| chaperone protein HscB, | | | | | | |
| mitochondrial | | | | | | |
| Interferon gamma | IFNGR1 | 5825-49 | P15260 | 9.077382 | 8.912255 | 8.999147 |
| receptor 1 | | | | | | |
| Insulin growth factor-like | IGFL3 | 6961-14 | Q6UXB1 | 11.27402 | 11.095608 | 11.185061 |
| family member 3 | | | | | | |
| Interleukin-10 receptor | IL10RA | 10344-334 | Q13651 | 11.426424 | 11.21058 | 11.296148 |
| subunit alpha | | | | | | |
| Interleukin-12 | IL-12A/ | 10367-62 | P29459/ | 7.654047 | 7.821953 | 7.742308 |
| | IL-12B | | P29460 | | | |
| Interleukin-21 | IL-21 | 7124-18 | Q9HBE4 | 10.178443 | 10.500567 | 10.212854 |
| Interleukin-7 | IL-7 | 4140-3 | P13232 | 7.567727 | 7.670461 | 7.627091 |
| Inter-alpha-trypsin | ITIH4 | 4811-33 | Q14624 | 15.196759 | 15.021511 | 15.172203 |
| inhibitor heavy chain H4 | | | | | | |
| Kelch-like protein 40 | KLHL40 | 24256-7 | Q2TBA0 | 8.511974 | 8.650932 | 8.599922 |
| Kallikrein-13 | KLK13 | 3200-49 | Q9UKR3 | 10.376438 | 10.255654 | 10.312592 |
| Kallikrein-14 | KLK14 | 3681-87 | Q9P0G3 | 14.620797 | 14.373216 | 14.521413 |
| Kininogen, HMW, Two | KNG1 | 15343-337 | P01042 | 12.297843 | 12.832776 | 12.174354 |
| Chain | | | | | | |
| Kininogen, HMW | KNG1 | 4918-21 | P01042 | 17.530925 | 17.524522 | 17.557302 |
| Kininogen, HMW | KNG1 | 7784-1 | P01042 | 11.813066 | 11.514945 | 11.600344 |
| Kininostatin | KNG1 | 19631-13 | P01042 | 12.916271 | 13.908774 | 13.072755 |
| Low-density lipoprotein | LDLR | 13129-40 | P01130 | 7.390888 | 7.518657 | 7.477497 |
| receptor | | | | | | |
| Liver-expressed | LEAP2 | 5708-1 | Q969E1 | 13.916145 | 13.162645 | 13.750683 |
| antimicrobial peptide 2 | | | | | | |
| Vesicular integral- | LMAN2 | 7638-30 | Q12907 | 12.727242 | 13.061726 | 12.818749 |
| membrane protein VIP36 | | | | | | |
| Low-density lipoprotein | LRP8 | 3323-37 | Q14114 | 10.669952 | 10.469686 | 10.553351 |
| receptor-related protein 8 | | | | | | |
| U6 snRNA-associated | LSM4 | 16854-17 | Q9Y4Z0 | 10.829965 | 10.681871 | 10.766737 |
| Sm-like protein LSm4 | | | | | | |
| Tumor necrosis factor | LTBR | 2636-10 | P36941 | 13.193116 | 12.832836 | 13.022485 |
| receptor superfamily | | | | | | |
| member 3 | | | | | | |
| T-lymphocyte surface | LY9 | 3324-51 | Q9HBG7 | 11.147668 | 10.86358 | 10.98456 |
| antigen Ly-9 | | | | | | |
| Melanoma-associated | MAGEA4 | 10485-56 | P43358 | 4.489174 | 5.127416 | 4.908359 |
| antigen 4 | | | | | | |
| Dual specificity mitogen- | MAP2K5 | 22041-26 | Q13163 | 13.229686 | 12.700952 | 13.08487 |
| activated protein kinase 5 | | | | | | |
| Homeobox protein MOX- | MEOX2 | 18259-15 | P50222 | 9.86519 | 10.04318 | 9.897547 |
| 2 | | | | | | |
| Matrix extracellular | MEPE | 3209-69 | Q9NQ76 | 7.406752 | 7.602635 | 7.498722 |
| phosphoglycoprotein | | | | | | |
| Methionine | METAP2 | 3170-6 | P50579 | 12.463558 | 12.016288 | 12.261264 |
| aminopeptidase 2 | | | | | | |
| Multimerin-2 | MMRN2 | 9723-105 | Q9H8L6 | 8.919683 | 8.76332 | 8.841154 |
| Mortality factor 4-like | MORF4L2 | 24443-8 | Q15014 | 10.57242 | 10.756498 | 10.594476 |
| protein 2 | | | | | | |
| 39S ribosomal protein | MRPL28 | 21123-1 | Q13084 | 8.382251 | 8.544633 | 8.478953 |
| L28, mitochondrial | | | | | | |
| S-methyl-5′-thioadenosine | MTAP | 9910-9 | Q13126 | 9.289234 | 9.40824 | 9.363933 |
| phosphorylase | | | | | | |
| NGFI-A-binding protein | NAB1 | 13933-276 | Q13506 | 11.535433 | 11.84743 | 11.732666 |
| 1 | | | | | | |
| NADH dehydrogenase | NDUFB4 | 10677-9 | O95168 | 11.062342 | 10.813155 | 10.979689 |
| [ubiquinone] 1 beta | | | | | | |
| subcomplex subunit 4 | | | | | | |
| U8 snoRNA-decapping | NUDT16 | 20948-16 | Q96DE0 | 10.908873 | 10.388972 | 10.88021 |
| enzyme | | | | | | |
| Olfactomedin-like protein | OLFML3 | 8660-33 | Q9NRN5 | 12.648785 | 12.876667 | 12.705647 |
| 3 | | | | | | |
| Oncostatin-M | OSM | 2693-20 | P13725 | 11.415415 | 11.140866 | 11.258277 |
| Succinyl-CoA: 3-ketoacid- | OXCT1 | 25043-74 | P55809 | 12.547084 | 12.175543 | 12.402845 |
| coenzyme A transferase | | | | | | |
| 1, mitochondrial | | | | | | |
| Poly(rC)-binding protein | PCBP2 | 23363-41 | Q15366 | 11.212458 | 10.428528 | 11.176152 |
| 2 | | | | | | |
| Protocadherin gamma-C3 | PCDHGC3 | 7859-21 | Q9UN70 | 11.306727 | 11.78872 | 11.555382 |
| 6-phosphogluconate | PGD | 4187-49 | P52209 | 11.972589 | 11.327932 | 11.787335 |
| dehydrogenase, | | | | | | |
| decarboxylating | | | | | | |
| Phosphatidylinositol | PITPNB | 12484-67 | P48739 | 11.012414 | 10.867485 | 10.941932 |
| transfer protein beta | | | | | | |
| isoform | | | | | | |
| Plexin domain-containing | PLXDC1 | 9235-3 | Q8IUK5 | 13.867907 | 13.430343 | 13.923705 |
| protein 1 | | | | | | |
| DNA polymerase epsilon | POLE2 | 11562-9 | P56282 | 8.581695 | 8.711129 | 8.660921 |
| subunit 2 | | | | | | |
| Calcineurin subunit B | PPP3R2 | 18866-8 | Q96LZ3 | 10.170243 | 10.00411 | 10.066986 |
| type 2 | | | | | | |
| Protein arginine N- | PRMT1 | 23351-59 | Q99873 | 11.296411 | 11.016773 | 11.188123 |
| methyltransferase 1 | | | | | | |
| Proteasome subunit beta | PSMB6 | 10530-8 | P28072 | 8.429878 | 8.555033 | 8.494029 |
| type-6 | | | | | | |
| Ras-related protein Rab- | RAB17 | 11118-107 | Q9H0T7 | 12.470066 | 12.231531 | 12.36014 |
| 17 | | | | | | |
| Ras-related protein Rab- | RAB4B | 23329-52 | P61018 | 11.411843 | 10.606958 | 11.251113 |
| 4B | | | | | | |
| Ras-related protein Rab- | RAB8B | 20167-6 | Q92930 | 9.320015 | 8.58169 | 9.117531 |
| 8B | | | | | | |
| DNA repair protein | RAD51 | 2871-73 | Q06609 | 11.761222 | 11.429489 | 11.562576 |
| RAD51 homolog 1 | | | | | | |
| GTP-binding nuclear | RAN | 4282-3 | P62826 | 10.951228 | 10.157625 | 10.687523 |
| protein Ran | | | | | | |
| Tumor necrosis factor | RELT | 5115-31 | Q969Z4 | 10.029663 | 9.859546 | 9.922363 |
| receptor superfamily | | | | | | |
| member 19L | | | | | | |
| 40S ribosomal protein | RPS12 | 21829-8 | P25398 | 12.621943 | 12.446434 | 12.575639 |
| S12 | | | | | | |
| 40S ribosomal protein S5 | RPS5 | 19195-85 | P46782 | 11.141587 | 10.928547 | 11.003482 |
| GTP-binding protein | SAR1B | 21660-4 | Q9Y6B6 | 11.989136 | 11.837767 | 11.905268 |
| SAR1b | | | | | | |
| Protein sel-1 homolog 2 | SEL1L2 | 9082-25 | Q5TEA6 | 10.660405 | 10.553649 | 10.620682 |
| Plasma serine protease | SERPINA5 | 3389-7 | P05154 | 15.779462 | 15.157215 | 15.808486 |
| inhibitor | | | | | | |
| Corticosteroid-binding | SERPINA6 | 4785-30 | P08185 | 8.448044 | 8.574976 | 8.519155 |
| globulin | | | | | | |
| Histone-lysine N- | SETD2 | 12647-52 | Q9BYW2 | 7.533134 | 7.640942 | 7.586677 |
| methyltransferase SETD2 | | | | | | |
| SH2 domain-containing | SHD | 23554-2 | Q96IW2 | 11.577231 | 11.362175 | 11.48603 |
| adapter protein D | | | | | | |
| Protein shisa-2 homolog | SHISA2 | 10449-31 | Q6UWI4 | 11.232809 | 11.108236 | 11.198411 |
| Sialic acid-binding Ig-like | SIGLEC12 | 8352-26 | Q96PQ1 | 11.102463 | 11.321204 | 11.186869 |
| lectin 12: Ig-like C2-type | | | | | | |
| 2 domain, Isoform short | | | | | | |
| Solute carrier family 35 | SLC35G2 | 13501-10 | Q8TBE7 | 10.969821 | 11.296492 | 11.084413 |
| member G2 | | | | | | |
| Stannin | SNN | 7762-30 | O75324 | 7.41218 | 8.026266 | 7.653731 |
| Alpha-N- | ST6GALNAC2 | 7823-22 | Q9UJ37 | 11.54395 | 11.277514 | 11.394306 |
| acetylgalactosaminide | | | | | | |
| alpha-2,6-sialyltransferase | | | | | | |
| 2 | | | | | | |
| Alpha-N- | ST6GALNAC3 | 10705-14 | Q8NDV1 | 7.836434 | 7.997283 | 7.933656 |
| acetylgalactosaminide | | | | | | |
| alpha-2,6-sialyltransferase | | | | | | |
| 3 | | | | | | |
| Serine/threonine-protein | STK16 | 3471-49 | O75716 | 10.809846 | 10.636156 | 10.713715 |
| kinase 16 | | | | | | |
| Sulfotransferase 1A3 | SULT1A3 | 13944-3 | P0DMM9 | 9.092898 | 8.711052 | 9.014951 |
| Heterogeneous nuclear | SYNCRIP | 4224-7 | O60506 | 12.010347 | 11.689539 | 11.866591 |
| ribonucleoprotein Q | | | | | | |
| Synaptotagmin-like | SYTL1 | 12892-10 | Q8IYJ3 | 8.928414 | 9.04689 | 8.982228 |
| protein 1 | | | | | | |
| Protein FAM19A4 | TAFA4 | 6511-17 | Q96LR4 | 9.910171 | 10.11183 | 9.929493 |
| Testis-expressed protein | TEX30 | 22802-13 | Q5JUR7 | 9.155261 | 9.322301 | 9.175297 |
| 30 | | | | | | |
| Enteropeptidase | TMPRSS15 | 3189-61 | P98073 | 8.58825 | 8.703585 | 8.665653 |
| Tropomyosin beta chain | TPM2 | 4472-5 | P07951 | 11.638375 | 11.281748 | 11.429319 |
| TLR4 interactor with | TRIL | 6527-1 | Q7L0X0 | 10.918794 | 10.747593 | 10.820367 |
| leucine rich | | | | | | |
| repeats: Extracellular | | | | | | |
| domain | | | | | | |
| Tripartite motif- | TRIM5 | 21438-45 | Q9C035 | 11.336743 | 11.753541 | 11.526622 |
| containing protein 5 | | | | | | |
| Ubiquitin-conjugating | UBE2R2 | 21848-18 | Q712K3 | 13.226217 | 13.009427 | 13.16064 |
| enzyme E2 R2 | | | | | | |
| Ubiquitin-protein ligase | UBE3A | 11226-16 | Q05086 | 11.259975 | 11.033459 | 11.20914 |
| E3A | | | | | | |
| Ubiquitin conjugation | UBE4A | 25073-13 | Q14139 | 11.747287 | 12.074219 | 11.802913 |
| factor E4 A | | | | | | |
| Urotensin-2 | UTS2 | 6020-52 | O95399 | 12.248676 | 12.017185 | 12.156354 |
| Tyrosine--tRNA ligase, | YARS1 | 19270-26 | P54577 | 12.95047 | 12.400307 | 12.984154 |
| cytoplasmic | | | | | | |
| Zinc fingers and | ZHX2 | 24955-116 | Q9Y6X8 | 7.522832 | 7.650393 | 7.597042 |
| homeoboxes protein 2 | | | | | | |
| Zinc finger protein 276 | ZNF276 | 14692-3 | Q8N554 | 12.518299 | 12.199046 | 12.363283 |
| E3 ubiquitin-protein | ZNRF3 | 10390-21 | Q9ULT6 | 8.75044 | 8.912557 | 8.735477 |
| ligase ZNRF3 | | | | | | |

### Utilities of the Protein Biomarkers

Aspects of the present disclosure relate to methods for analyzing samples obtained from subjects (e.g., human patients) having, suspected of having, or being at risk for a disease or disorder, such as a disease or disorder associated with the contact activation system, by measuring the level of a biomarker set as described herein in the sample. In some aspects, the methods described herein relate to identifying subjects having or at risk of having a disorder associated with any of the proteins shown in Table 1, and optionally, treating the subject with a plasma kallikrein inhibitor. Yet other aspects of the present disclosure relate to determining if a disorder is susceptible to treatment with a pKal inhibitor and evaluating the efficacy of a treatment of a disorder in a subject. Results obtained from the methods described herein may be useful for diagnostic and/or prognostic purposes, as well as for other non-clinical purposes, such as research purposes.

### (i) Analysis of Biological Samples

The methods described herein involved providing a biological sample obtained from a subject. As used herein, a “biological sample” refers to a composition that comprises tissue, e.g., blood, plasma, or protein, from a subject. A sample includes both an initial unprocessed sample taken from a subject as well as subsequently processed, e.g., partially purified or preserved forms. Exemplary samples include blood, plasma, tears, or mucus. In some embodiments, the sample is a body fluid sample such as a serum or plasma sample. In some embodiments, multiple (e.g., at least 2, 3, 4, 5, or more) biological samples may be collected from subject, over time or at particular time intervals, for example to assess the disease progression or evaluate the efficacy of a treatment. In some embodiments, multiple (e.g., at least 2, 3, 4, 5, or more) biological samples may be collected from subject, for example prior to treatment, during treatment, and/or after treatment to assess the disease progression or evaluate the efficacy of a treatment. In some embodiments, the biological sample is plasma.

A biological sample can be obtained from a subject using any means known in the art. In some embodiments, the sample is obtained from the subject by collecting the sample (e.g., a blood sample) into an evacuated collection tube (e.g., an evacuated blood collection tube). In some embodiments, the evacuated collection tube contains one or more protease inhibitors, for example, to reduce or prevent ex vivo activation of the contact system during sample collection. Such protease inhibitors may be contained in a liquid formulation. In some embodiments, the protease inhibitors comprise at least one serine protease inhibitor and at least one cysteine protease inhibitor. Such evacuated collection tubes are known in the art. See, for example, PCT Application No. PCT/US2016/046681 (WO 2017/027771), which is incorporated by reference herein in its entirety. Optionally, an evacuated blood collection tube may further comprise one or more anti-coagulants.

The terms “patient,” “subject,” or “individual” may be used interchangeably and refer to a subject as described herein. In some embodiments, the subject is a human or a non-human mammal. In some embodiments, a subject is suspected of or is at risk for a disease or disorder, such as a disease or disorder involving or mediated by any of the proteins in Table 1. In some embodiments, a subject is suspected of or is at risk for a disease or disorder associated with the contact activation system (e.g., HAE). Such a subject may exhibit one or more symptoms associated with the disease. Alternatively or in addition, such a subject may carry one or more risk factors for the disease, for example, a genetic factor associated with the disease (e.g., a genetic defect in CI-INH).

Alternatively, the subject described herein may be a patient of the disease. Such a subject may be in an acute stage of the disease currently (e.g., experiencing a HAE attack), or may suffer from the disease in the past (e.g., during disease quiescence currently (baseline)). In some examples, the subject is a human patient who may be on a treatment of the disease, for example, a treatment involving an agent that targets the kallikrein-kinin system (KKS), such as a C1 esterase inhibitor (C1-INH), a plasma kallikrein inhibitor, or a bradykinin inhibitor. In other instances, such a human patient may be free of such a treatment.

The methods described herein relate to determining if a disorder is susceptible to treatment with a pKal inhibitor. In such embodiments, the disease or disorder may not have previously been associated with pKal activity and/or the contact activation system. Such methods may be used to identify additional diseases or disorders for which treatment with a pKal inhibitor may be effective. In some embodiments, the disease or disorder is associated with or mediated by aberrant expression or activity of any one or more of the proteins shown in Table 1. In some embodiments, the disease or disorder is not associated with the contact activation system. In some embodiments, the disease or disorder is not hereditary angioedema.

In some embodiments, the disease or disorder is associated with the contact activation system. Examples of diseases associated with the contact activation system include, without limitation, kallikrein-mediated disorders, e.g., a bradykinin-mediated disorder, such as hereditary angioedema (HAE), non-histamine-dependent idiopathic angioedema, rheumatoid arthritis, Crohn's disease, lupus, Alzheimer's disease, septic shock, burn injury, brain ischemia/reperfusion injury, cerebral edema, diabetic retinopathy, diabetic nephropathy, macular edema, vasculitis, arterial or venous thrombosis, thrombosis associated with ventricular assist devices or stents, heparin-induced thrombocytopenia with thrombosis, thromboembolic disease, and coronary heart disease with unstable angina pectoris, edema, eye disease, gout, intestinal bowel disease, oral mucositis, neuropathic pain, inflammatory pain, spinal stenosis-degenerative spine disease, post-operative ileus, aortic aneurysm, osteoarthritis, hereditary angioedema, pulmonary embolism, stroke, head trauma or peri-tumor brain edema, sepsis, acute middle cerebral artery (MCA) ischemic event (stroke), restenosis (e.g., after angioplasty), systemic lupus erythematosis nephritis, an autoimmune disease, an inflammatory disease, a cardiovascular disease, a neurological disease, a disease associated with protein misfolding, a disease associated with angiogenesis, hypertensive nephropathy and diabetic nephropathy, allergic and respiratory diseases (e.g., anaphylaxis, asthma, chronic obstructive pulmonary disease, acute respiratory distress syndrome, cystic fibrosis, persistent, rhinitis), and tissue injuries (e.g., burn or chemical injury).

In some embodiments, the disease or disorder that is associated with the contact activation system is hereditary angioedema (HAE). In some embodiments, the methods described herein may be used to identify whether a subject has or is at risk of having HAE. Alternatively or in addition, the methods described herein may be used to evaluate efficacy of a treatment and/or disease progression. Alternatively or in addition, the methods described herein may be used to evaluate whether a subject has or is at risk of having an HAE attack. In some embodiments, the methods described herein may be used to determine whether to administer one or more additional doses of a therapeutic agent, e.g., a pKal inhibitor, modulate the dosage of a therapeutic agent, e.g., increase or decrease a dosage amount and/or frequency or initiate or terminate a treatment for HAE. Hereditary angioedema (HAE) is also known as “Quincke edema,” C1 esterase inhibitor deficiency, C1 inhibitor deficiency, and hereditary angioneurotic edema (HANE). HAE is characterized by recurrent episodes of severe swelling (angioedema), which can affect, e.g., the limbs, face, genitals, gastrointestinal tract, and airway. Symptoms of HAE include, e.g., swelling in the arms, legs, lips, eyes, tongue, and/or throat; airway blockage that can involve throat swelling and sudden hoarseness; repeat episodes of abdominal cramping without obvious cause; and/or swelling of the intestines, which can be severe and can lead to abdominal cramping, vomiting, dehydration, diarrhea, pain, and/or shock. About one-third of individuals with HAE develop a non-itchy rash called erythema marginatum during an attack.

Swelling of the airway can be life threatening and causes death in some patients. Mortality rates are estimated at 15-33%. HAE leads to about 15,000-30,000 emergency department visits per year.

Trauma or stress, e.g., dental procedures, sickness (e.g., viral illnesses such as colds and the flu), menstruation, and surgery can trigger an attack of angioedema. To prevent acute attacks of HAE, patients can attempt to avoid specific stimuli that have previously caused attacks. However, in many cases, an attack occurs without a known trigger. Typically, HAE symptoms first appear in childhood and worsen during puberty. On average, untreated individuals have an attack every 1 to 2 weeks, and most episodes last for about 3 to 4 days (ghr.nlm.nih.gov/condition/hereditary-angioedema). The frequency and duration of attacks vary greatly among people with hereditary angioedema, even among people in the same family.

There are three types of HAE, known as types I, II, and III. It is estimated that HAE affects 1 in 50,000 people, that type I accounts for about 85 percent of cases, type II accounts for about 15 percent of cases, and type III is very rare. Type III is the most newly described form and was originally thought to occur only in women, but families with affected males have been identified.

HAE is inherited in an autosomal dominant pattern, such that an affected person can inherit the mutation from one affected parent. New mutations in the gene can also occur, and thus HAE can also occur in people with no history of the disorder in their family. It is estimated that 20-25% of cases result from a new spontaneous mutation.

Mutations in the SERPING1 gene cause hereditary angioedema type I and type II. The SERPING1 gene provides instructions for making the C1 inhibitor protein, which is important for controlling inflammation. C1 inhibitor blocks the activity of certain proteins that promote inflammation. Mutations that cause hereditary angioedema type I lead to reduced levels of C1 inhibitor in the blood. In contrast, mutations that cause type II result in the production of a C1 inhibitor that functions abnormally. Without the proper levels of functional C1 inhibitor, excessive amounts of bradykinin are generated. Bradykinin promotes inflammation by increasing the leakage of fluid through the walls of blood vessels into body tissues. Excessive accumulation of fluids in body tissues causes the episodes of swelling seen in individuals with hereditary angioedema type I and type II.

Mutations in the F12 gene are associated with some cases of hereditary angioedema type III. The F12 gene provides instructions for making coagulation factor XII. In addition to playing a critical role in blood clotting (coagulation), factor XII is also an important stimulator of inflammation and is involved in the production of bradykinin. Certain mutations in the F12 gene result in the production of factor XII with increased activity. As a result, more bradykinin is generated and blood vessel walls become more leaky, which leads to episodes of swelling. The cause of other cases of hereditary angioedema type III remains unknown. Mutations in one or more as-yet unidentified genes may be responsible for the disorder in these cases.

HAE can present similarly to other forms of angioedema resulting from allergies or other medical conditions, but it differs significantly in cause and treatment. When HAE is misdiagnosed as an allergy, it is most commonly treated with antihistamines, steroids, and/or epinephrine, which are typically ineffective in HAE, although epinephrine can be used for life-threatening reactions. Misdiagnoses have also resulted in unnecessary exploratory surgery for patients with abdominal swelling, and in some HAE patients abdominal pain has been incorrectly diagnosed as psychosomatic.

C1 inhibitor therapies, as well as other therapies for HAE, are described in Kaplan, A. P., J Allergy Clin Immunol, 2010, 126 (5): 918-925.

Acute treatment of HAE attacks is provided to halt progression of the edema as quickly as possible. C1 inhibitor concentrate from donor blood, which is administered intravenously, is one acute treatment; however, this treatment is not available in many countries. In emergency situations where C1 inhibitor concentrate is not available, fresh frozen plasma (FFP) can be used as an alternative, as it also contains C1 inhibitor.

Purified C1 inhibitor, derived from human blood, has been used in Europe since 1979. Several C1 inhibitor treatments are now available in the U.S. and two C1 inhibitor products are now available in Canada. Berinert P (CSL Behring), which is pasteurized, was approved by the F.D.A. in 2009 for acute attacks. CINRYZE® (Takeda), which is nanofiltered, was approved by the F.D.A. in 2008 for prophylaxis. Rhucin/Ruconest (Pharming) is a recombinant C1 inhibitor under development that does not carry the risk of infectious disease transmission due to human blood-borne pathogens.

Treatment of an acute HAE attack also can include medications for pain relief and/or IV fluids.

Other treatment modalities can stimulate the synthesis of C1 inhibitor, or reduce C1 inhibitor consumption. Androgen medications, such as danazol, can reduce the frequency and severity of attacks by stimulating production of C1 inhibitor.

Helicobacter pylori can trigger abdominal attacks. Antibiotics to treat H. pylori will decrease abdominal attacks.

Newer treatments attack the contact cascade. Ecallantide (KALBITOR®, Takeda) and lanadelumab (TAKHZYRO®, Takeda) inhibit plasma kallikrein and have been approved in the U.S., or both the U.S. and Europe, respectively. Icatibant (FIRAZYR®, Takeda) inhibits the bradykinin B2 receptor, and has been approved in Europe and the U.S.

Diagnosis of HAE can rely on, e.g., family history and/or blood tests. Laboratory findings associated with HAE types I, II, and III are described, e.g., in Kaplan, A. P., J Allergy Clin Immunol, 2010, 126 (5): 918-925. In type I HAE, the level of C1 inhibitor is decreased, as is the level of C4, whereas C1q level is normal. In type II HAE, the level of C1 inhibitor is normal or increased; however, C1 inhibitor function is abnormal. C4 level is decreased and C1q level is normal. In type III, the levels of C1 inhibitor, C4, and C1q can all be normal. The present disclosure is based, at least in part, on the identification of additional proteins that have differential levels in samples from HAE patients as compared to healthy individuals (Table 1). Measuring the levels of biomarker sets of these proteins can be used to identify whether a subject has a disease, such as HAE. In some embodiments, the methods may be used to determine whether a patient has had or is having an HAE attack. In some embodiments, the methods may be used to determine whether a treatment is or has been effective in treating HAE.

Symptoms of a disease or disorder, such as HAE, can be assessed, for example, using questionnaires, e.g., questionnaires that are completed by patients, clinicians, or family members. Such questionnaires are known in the art and include, for example, visual analog scales. See, e.g., McMillan, C. V. et al. Patient (2012) 5 (2): 113-26.

The biological sample described herein can be subject to analysis by measuring the level of a biomarker set as described herein in the biological sample. Levels (e.g., the amount) of a biomarker disclosed herein, or changes in levels the biomarker, can be assessed using assays described herein and/or assays known in the art. One or more of the biomarkers described herein may be analyzed using convention methods. In some embodiments, the level of a biomarker is assessed or measured by directly detecting the protein in a biological sample. Alternatively or in addition, the level of a protein can be assessed or measured by indirectly in a biological sample, for example, by detecting the level of activity of the protein (e.g. enzymatic assay).

In some embodiments, the biomarker is measured using an immunoassay. Examples of immunoassays include, without limitation immunoblotting assays (Western blots), enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting a biomarker provided herein will be apparent to those of skill in the art. It will be apparent to those of skill in the art that this disclosure is not limited to immunoassays, however, and that detection assays that are not based on an antibody or an antigen binding antibody fragment, such as mass spectrometry, are also useful for the detection and/or quantification of biomarkers as provided herein. Assays that rely on a chromogenic substrate can also be useful for the detection and/or quantification of the biomarkers as provided herein.

The type of detection assay used for the detection and/or quantification of a biomarker, such as those provided herein, will depend on the particular situation in which the assay is to be used (e.g., clinical or research applications), and on the kind and number of biomarkers to be detected, and on the kind and number of patient samples to be run in parallel, to name a few parameters.

ELISAs are known in the art (see, e.g., Crowther, John R (2009). “The ELISA Guidebook.” 2nd ed. Humana Press and Lequin R (2005). “Enzyme immunoassay (EIA)/enzyme-linked immunosorbent assay (ELISA)” Clin. Chem. 51 (12): 2415-8) and exemplary ELISAs are described herein. Kits for performing ELISAs are also known in the art and commercially available (see, e.g., ELISA kits from Life Technologies and BD Biosciences).

In some embodiments, an immunoassay is used to measure levels of the protein biomarker(s). The immunoassays described herein may be in the format of a sandwich ELISA, in which a first binding agent that specifically binds a protein of the biomarker set is immobilized on a support member. The support member can then be incubated with a biological sample as described herein for a suitable period of time under conditions that allow for the formation of complex between the binding agent and the protein in the sample. Such a complex can then be detected using a detection agent that binds the protein, the binding agent-protein complex, or the binding agent. The detection agent can be conjugated to a label, which can release a signal directly or indirectly. The intensity of the signal represents the level of the protein in the sample. In some embodiments, the detection agent is detected and its level represents the level of the protein in the sample.

Any binding agent that specifically binds to a desired protein may be used in the methods and kits described herein to measure the level of a protein in a biological sample. In some embodiments, the binding agent is an antibody that specifically binds to a desired protein. In some embodiments, the binding agent is an aptamer that specifically binds to a desired protein. In some embodiments, a sample may be contacted, simultaneously or sequentially, with more than one binding agent that bind different proteins (e.g., multiplexed analysis, for example the SOMASCAN® assay (SOMALogic)). The biological sample is contacted with a binding agent under appropriate conditions. In general, the term “contact” refers to an exposure of the binding agent with the biological sample or agent for a suitable period sufficient for the formation of complexes between the binding agent and the protein in the sample, if any. In some embodiments, the contacting is performed by capillary action in which a biological sample or agent is moved across a surface of the support membrane.

In some embodiments, the immunoassays may be performed on low-throughput platforms, including in single immunoassay format. For example, a low throughput platform may be used to measure the presence and amount of a protein in biological samples (e.g., biological tissues, tissue extracts) for diagnostic methods, monitoring of disease and/or treatment progression, and/or predicting whether a disease or disorder may benefit from a particular treatment.

In some embodiments, it may be necessary to immobilize a binding agent to the support member. Methods for immobilizing a binding agent will depend on factors such as the nature of the binding agent and the material of the support member and may require particular buffers. Such methods will be evident to one of ordinary skill in the art. For example, the biomarker set in a biological sample as described herein may be measured using any of the kits and/or detecting devices which are also described herein.

As used herein, the terms “measuring” or “measurement,” or alternatively “detecting” or “detection,” means assessing the presence, absence, quantity or amount (which can be an effective amount) of a substance within a sample, including the derivation of qualitative or quantitative concentration levels of such substances, or otherwise evaluating the values or categorization of a subject.

Assays, e.g., Western blot assays, may further involve use of a quantitative imaging system, e.g., LICOR imaging technology, which is commercially available (see, e.g., the Odyssey® CLx infrared imaging system from LI-COR Biosciences). In some embodiments, an electrochemiluminescence detection assay or an assay relying on a combination of electrochemiluminescence and patterned array technology is used (e.g., an ECL or MULTI-ARRAY technology assay from Meso Scale Discovery (MSD)).

In any of the methods described herein, the level of protein of a biomarker set can be compared to the level of the protein in a control sample or a reference sample. Alternatively or in addition, in any of the methods described herein, the level of protein of a biomarker set can be compared to a reference level, such as a predetermined reference level.

### (ii) Clinical Applications

The levels of proteins presented in Table 1 detected in samples from subjects can be used as biomarkers for identifying subjects as having or being at risk of having a disease or disorder, diagnosing diseases, such as diseases associated with the contact activation system (e.g., HAE), monitoring the progress of a disease, assessing the efficacy of a treatment for the disease, identifying patients suitable for a particular treatment.

Accordingly, described herein are diagnostic and prognostic methods for a disease or disorder, such as a disease or disorder associated with expression of any of the proteins shown in Table 1, based on the level of a biomarker set in a biological sample obtained from a subject. In some embodiments, a disease or disorder associated with expression of any of the proteins shown in Table 1 may be treated with an agent, such as lanadelumab. In some embodiments, the methods are diagnostic and prognostic methods for a disease associated with the contact activation system based on the level of a biomarker set in a biological sample obtained from a subject.

In some embodiments, the level of the biomarker, as measured using any of the methods described herein, can be relied on to evaluate whether a subject (e.g., a human patient) from whom the biological sample is obtained, has or is at risk for a disease. In some embodiments, the level of the biomarker, as measured using any of the methods described herein, can be relied on to evaluate whether a subject (e.g., a human patient) from whom the biological sample is obtained, has or is at risk for a disease associated with the contact activation system, such as a disease associated with plasma kallikrein, e.g., HAE or autoimmune disease such as RA, UC, and Crohn's disease. In some embodiments, the methods are diagnostic and prognostic methods for a disease that may be mediated by or associated with the kallikrein-kinin system (contact activation system) and therefore, may be treated with an agent that targets the kallikrein-kinin system (e.g., lanadelumab).

In some embodiments, the level of the biomarker can then be compared with a reference sample or a control sample to determine a value indicating the amount of the protein in the sample. In some embodiments, a value for a biomarker is obtained by comparing the level of a protein in a sample to the level of another protein (e.g., an internal control or internal standard) in the sample. Such a biomarker value may be a normalized value over the internal control or internal standard. The value of the biomarker can be compared to a reference value to determine whether the subject has or is at risk for the disease. The reference value may represent the level of the corresponding biomarker in subjects (e.g., human subjects) free of the target disease (e.g., healthy subject). In some embodiments, if the level or value of the biomarker is higher than a reference level or value, the subject can be identified as having or at risk for a disease. In some embodiments, if the level or value of the biomarker is lower than a reference level or value, the subject can be identified as having or at risk for a disease.

In some embodiments, the level of the biomarker can be compared to a predetermined threshold for the protein, a deviation from which may indicate the subject has a disease. The predetermined threshold may represent the value of the biomarker that distinguishes the level of the biomarker in patients having the target disease from the level of the biomarker in patients free of the target disease.

In some embodiments, the biomarker set includes more than one protein, for at least one of which an elevated level indicates the subject has or is at risk of having the disease and for at least one of the proteins a reduced level indicates the subject has or is at risk of having the disease. In some embodiments, the biomarker set includes more than one protein, for each of which an elevated level indicates the subject has or is at risk of having the disease. In some embodiments, the biomarker set includes more than one protein, for each of which a reduced level indicates the subject has or is at risk of having the disease.

In some embodiments, the control sample or reference sample is a biological sample obtained from a healthy individual. In some embodiments, the control sample or reference sample contains a known amount of the protein to be assessed. In some embodiments, the control sample or reference sample is a biological sample obtained from a control subject.

As used herein, a control subject may be a healthy individual, i.e., an individual that is apparently free of the target disease (e.g., a disease associated with the contact system) at the time the level of the protein(s) is measured or has no history of the disease. A control subject may also represent a population of healthy subjects, who preferably would have matches features (e.g., age, gender, ethnic group) as the subject being analyzed by a method described herein.

The control level can be a predetermined level or threshold. Such a predetermined level can represent the level of the protein in a population of subjects that do not have or are not at risk for the target disease (e.g., the average level in the population of healthy subjects). It can also represent the level of the protein in a population of subjects that have the target disease.

The predetermined level can take a variety of forms. For example, it can be single cut-off value, such as a median or mean. In some embodiments, such a predetermined level can be established based upon comparative groups, such as where one defined group is known to have a target disease and another defined group is known to not have the target disease. Alternatively, the predetermined level can be a range, for example, a range representing the levels of the protein in a control population.

The control level as described herein can be determined by routine technology. In some examples, the control level can be obtained by performing a conventional method (e.g., the same assay for obtaining the level of the protein a test sample as described herein) on a control sample as also described herein. In other examples, levels of the protein can be obtained from members of a control population and the results can be analyzed by, e.g., a computational program, to obtain the control level (a predetermined level) that represents the level of the protein in the control population.

By comparing the level of a biomarker in a sample obtained from a candidate subject to the reference value as described herein, it can be determined as to whether the candidate subject has or is at risk for a disease. For example, if the level of biomarker(s) in a sample of the candidate subject deviates from the reference value (e.g., increased or decreased as compared to the reference value), the candidate subject might be identified as having or at risk for the disease. When the reference value represents the value range of the level of the biomarker in a population of subjects that have the target disease, the value of biomarker in a sample of a candidate falling in the range indicates that the candidate subject has or is at risk for the target disease.

As used herein, “an elevated level” or “a level above a reference value” means that the level of the biomarker is higher than a reference value, such as a pre-determined threshold of a level the biomarker in a control sample. Control levels are described in detail herein. An elevated level of a biomarker includes a level of the biomarker that is, for example, 1%, 5%, 10%, 20%, 30%, 40%, 50%, 60%, 70%, 80%, 90%, 100%, 150%, 200%, 300%, 400%, 500% or more above a reference value. In some embodiments, the level of the biomarker in the test sample is at least 1.1., 1.2, 1.3, 1.4, 15, 1.6, 1.7, 1.8, 1.9, 2, 2.5, 3, 3.5, 4, 4.5, 5, 5.5, 6, 7, 8, 9, 10, 50, 100, 150, 200, 300, 400, 500, 1000, 10000-fold or higher than the level of the biomarker in a reference sample.

As used herein, “a decreased level” or “a level below a reference value” means that the level of the biomarker is lower than a reference value, such as a pre-determined threshold of the biomarker in a control sample. Control levels are described in detail herein. A decreased level of the biomarker includes a level of the biomarker that is, for example, 1%, 5%, 10%, 20%, 30%, 40%, 50%, 60%, 70%, 80%, 90%, 100%, 150%, 200%, 300%, 400%, 500% or more lower than a reference value. In some embodiments, the level of the biomarker in the test sample is at least 1.1., 1.2, 1.3, 1.4, 15, 1.6, 1.7, 1.8, 1.9, 2, 2.5, 3, 3.5, 4, 4.5, 5, 5.5, 6, 7, 8, 9, 10, 50, 100, 150, 200, 300, 400, 500, 1000, 10000-fold or lower than the level of the biomarker in a reference sample.

In some embodiments, the methods described herein are for identifying a subject as a candidate for a particular treatment, e.g., treatment with a pKal inhibitor. In some embodiments, the candidate subject is a human patient having a symptom of a disease, such as a disease associated with the contact activation system, such as a pKal-mediated disorder, e.g., HAE or an autoimmune disease such as RA, UC, and Crohn's disease. For example, the subject has edema, swelling wherein said swelling is completely or predominantly peripheral; hives; redness, pain, and swelling in the absence of evidence of infection; non-histamine-mediated edema, recurrent attacks of swelling, or a combination thereof. In other embodiments, the subject has no symptom of a pKal-mediated disorder at the time the sample is collected, has no history of a symptom of a pKal-mediated disorder, or no history of a pKal-mediated disorder such as HAE. In yet other embodiments, the subject is resistant to an anti-histamine therapy, a corticosteroid therapy, or both.

A subject identified in the methods described herein may be subject to a suitable treatment, such as treatment with a pKal inhibitor, as described herein.

The assay methods and kits described herein also can be applied for evaluation of the efficacy of a treatment with a pKal inhibitor, such as those described herein, given the correlation between the level of the biomarkers and such diseases. For examples, multiple biological samples (e.g., blood or plasma samples) can be collected from a subject to whom a treatment is performed either before and after the treatment or during the course of the treatment. The levels of a biomarker can be measured by any of the assay methods as described herein and values (e.g., amounts) of a biomarker can be determined accordingly. For example, if an elevated level of a biomarker indicates that a subject has a target disease and the level of the biomarker decreases after the treatment or over the course of the treatment (the level of the biomarker in a later collected sample as compared to that in an earlier collected sample), it indicates that the treatment is effective. As another example, if a reduced level of a biomarker indicates that a subject has a target disease and the level of the biomarker increases after the treatment or over the course of the treatment (the level of the biomarker in a later collected sample as compared to that in an earlier collected sample), it indicates that the treatment is effective. A treatment may also be determined to be effective if the level of a biomarker or biomarker set in a subject (e.g., a biomarker or biomarker set that indicates disease) becomes normalized (e.g., does not deviate from the level of the same biomarker or biomarker set in a control subject, e.g., a healthy subject or a subject having the disease in a quiescent state) during or after a course of treatment. In some embodiments, the level of a biomarker or biomarker set does not substantially deviate from the level of the same biomarker or biomarker set in a control subject if there is not a statistically significant difference between the level of the biomarker or biomarker set and that of a control subject. As an example, an elevated biomarker that is indicative of disease prior to treatment may decrease to a level that is similar to (does not substantially deviate from) the level of the biomarker in a subject who does not have the disease, in which case the treatment is determined to be effective. In another example, a decreased biomarker that is indicative of disease prior to treatment may increase to a level that is similar to (does not substantially deviate from) the level of the biomarker in a subject who does not have the disease, in which case the treatment is determined to be effective. In some examples, the treatment involves one or more administrations of plasma kallikrein inhibitor, such as lanadelumab.

If the subject is identified as not responsive to the treatment, or the treatment is determined to not have been effective, a higher dose and/or frequency of dosage of the therapeutic agent (e.g., a pKal inhibitor, such as lanadelumab) are administered to the subject identified. In some embodiments, the dosage or frequency of dosage of the therapeutic agent (e.g., a pKal inhibitor, such as lanadelumab) is maintained, lowered, or ceased in a subject identified as responsive to the treatment or is not in need of further treatment. Alternatively, a different treatment can be applied to the subject who is found as not responsive to the first treatment.

In other embodiments, the values of a biomarker or biomarker set can also be used to identify that a disorder be susceptible (treatable) to treatment with a pKal inhibitor. To practice this method, the level of a biomarker in a sample collected from a subject (e.g., a blood sample or a plasma sample) having a target disease can be measured by a suitable method, e.g., those described herein such as a Western blot or ELISA assay. If the level of the biomarker deviates from the reference value (e.g., elevated or decreased), it indicates that a pKal inhibitor may be effective in treating the disease. If the disease is identified as being susceptible (can be treated by) to a pKal inhibitor, the method can further comprise administering to the subject having the disease an effective amount of a pKal inhibitor, such as an anti-pKal antibody (e.g., lanadelumab) or an inhibitory peptide (e.g., ecallantide); a bradykinin 2 receptor inhibitor (e.g., icatibant); and/or a C1-INH (e.g., human plasma-derived C1-INH).

(iii) Non-Clinical Applications

Further, levels of any of the biomarker set described herein may be used for research purposes. Although many diseases associated with the contact activation system have been identified, it is possible that other diseases are mediated by similar mechanisms or involve similar components, for example proteins whose levels are affected by lanadelumab treatment. In some embodiments, the methods described herein may be used to identify a disease as being associated with the contact activation system or with components of the contact activation system (e.g., pKal activity). In some embodiments, the methods described herein may be used to study mechanisms (e.g., the discovery of novel biological pathways or processes involved in disease development) or progression of a disease.

In some embodiments, the levels of biomarker sets, as described herein, may be used in the development of new therapeutics for a disease. For example, the levels of a biomarker set may be measured in samples obtained from a subject having been administered a new therapy (e.g., a clinical trial). In some embodiments, the level of the biomarker set may indicate the efficacy of the new therapeutic or the progression of the disease in the subject prior to, during, or after the new therapy.

### Methods of Treatment

Also within the scope of the present disclosure are methods of identifying subjects having or at risk of having a disorder, identifying subjects as candidates for treatment, and methods of treating subjects and disorders in a subject. A subject at risk for or suffering from a disorder, such as a disorder mediated by or associated with any of the biomarkers in Table 1, may be identified using the methods described herein, and may further be treated with any appropriate therapeutic agent. In some embodiments, provided methods include selecting a treatment for a subject based on the output of the described method, e.g., measuring the level of a biomarker set.

In some embodiments, the method comprises one or both of selecting or administering a therapeutic agent, e.g., a plasma kallikrein (pKal) inhibitor, a bradykinin B2 receptor inhibitor, and/or a C1 esterase inhibitor, for administration to the subject based on the output of the assay, e.g., biomarker detection/level.

In some embodiments, the therapeutic agent is administered one or more times to the subject. In some embodiments, a plasma kallikrein inhibitor is administered one or more times to a subject. In some embodiments, the pKal inhibitor is a peptide, a small molecule inhibitor, a kallikrein antibody, or a fragment thereof. In some embodiments, an antagonist of bradykinin B2 receptor is administered to a subject. In some embodiments, a C1 esterase inhibitor (C1-INH) is administered to a subject.

The therapeutic agent, e.g., a pKal inhibitor, bradykinin B2 receptor inhibitor, and/or C1-INH, may be administered along with another therapy as part of a combination therapy for treatment of the disease or condition that involves the contact activation system. Combination therapy, e.g., with one or more of a pKal inhibitor, bradykinin B2 receptor antagonist, or C1-INH replacement agent, e.g., with one or more of a pKal inhibitor, bradykinin B2 receptor antagonist or C1-INH replacement agent and another therapy, may be provided in multiple different configurations. The first agent may be administered before or after the administration of the other therapy. In some situations, the first agent and another therapy (e.g., a therapeutic agent) are administered concurrently, or in close temporal proximity (e.g., a short time interval between the injections, such as during the same treatment session). The first agent and the other therapy may also be administered at greater temporal intervals.

### Therapeutic Agents

Plasma kallikrein binding agents (e.g., binding proteins, e.g., polypeptides, e.g., inhibitory polypeptides, e.g., antibodies, e.g., inhibitory antibodies, or other binding agents, e.g., small molecules) are useful therapeutic agents for a variety of disorders and conditions, e.g., diseases and conditions associated with expression of the biomarkers of Table 1. In some embodiments, the disease or disorder is associated with the contact activation system, e.g., involving pKal activity. In some embodiments, the disease or condition that involves plasma kallikrein activity is hereditary angioedema (HAE). In some embodiments, the disease or condition is not hereditary angioedema. In some embodiments, a plasma kallikrein inhibitor is administered to a subject at risk or suffering from a disease associated with the contact activation system.

A number of useful protein inhibitors of kallikrein, either tissue and/or plasma kallikrein, include a Kunitz domain. As used herein, a “Kunitz domain” is a polypeptide domain having at least 51 amino acids and containing at least two, and preferably three, disulfides. The domain is folded such that the first and sixth cysteines, the second and fourth, and the third and fifth cysteines form disulfide bonds (e.g., in a Kunitz domain having 58 amino acids, cysteines can be present at positions corresponding to amino acids 5, 14, 30, 38, 51, and 55, according to the number of the BPTI homologous sequences provided below, and disulfides can form between the cysteines at position 5 and 55, 14 and 38, and 30 and 51), or, if two disulfides are present, they can form between a corresponding subset of cysteines thereof. The spacing between respective cysteines can be within 7, 5, 4, 3, 2, 1 or 0 amino acids of the following spacing between positions corresponding to: 5 to 55, 14 to 38, and 30 to 51, according to the numbering of the BPTI sequence provided below. The BPTI sequence can be used as a reference to refer to specific positions in any generic Kunitz domain. Comparison of a Kunitz domain of interest to BPTI can be performed by identifying the best fit alignment in which the number of aligned cysteines in maximized.

The 3D structure (at high resolution) of the Kunitz domain of BPTI is known. One of the X-ray structures is deposited in the Brookhaven Protein Data Bank as “6PTI”. The 3D structure of some BPTI homologues (Eigenbrot et al., Protein Engineering (1990) 3(7): 591-598; Hynes et al., Biochemistry (1990) 29:10018-10022) are known. At least eighty one Kunitz domain sequences are known. Known human homologues include three Kunitz domains of LACI also known as tissue factor pathway inhibitor (TFPI) (Wun et al., J. Biol. Chem. (1988) 263(13): 6001-6004; Girard et al., Nature (1989) 338:518-20; Novotny et al, J. Biol. Chem. (1989) 264(31): 18832-18837) two Kunitz domains of Inter-α-Trypsin Inhibitor, APP-I (Kido et al. J. Biol. Chem. (1988) 263(34): 18104-18107), a Kunitz domain from collagen, three Kunitz domains of TFPI-2 (Sprecher et al., PNAS USA (1994) 91:3353-3357), the Kunitz domains of hepatocyte growth factor activator inhibitor type 1, the Kunitz domains of Hepatocyte growth factor activator inhibitor type 2, the Kunitz domains described in U.S. Patent Publication No.: 2004-0152633. LACI is a human serum phosphoglycoprotein with a molecular weight of 39 kDa (amino acid sequence in Table 2) containing three Kunitz domains.

TABLE 2
Exemplary Natural Kunitz Domains

| | | |
|---|---|---|
| LACI | 1 | MIYTMKKVHA LWASVCLLLN LAPAPLNAds eedeehtiit dtelpplklM |
| (SEQ ID | 51 | HSFCAFKADD GPCKAIMKRF FFNIFTRQCE EFIYGGCEGN QNRFESLEEC |
| NO: 1) | 101 | KKMCTRDnan riikttlqqe kpdfCfleed pgiCrgyitr yfynnqtkqC |
| | 151 | erfkyggClg nmnnfetlee CkniCedgpn gfqvdnygtq lnavnnsltp |
| | 201 | qstkvpslfe fhgpswCltp adrglCrane nrfyynsvig kCrpfkysgC |
| | 251 | ggnennftsk qeClraCkkg fiqriskggl iktkrkrkkq rvkiayeeif |
| | 301 | vknm |

| | |
|---|---|
| | The signal sequence (1-28) is uppercase and underscored |
| | LACI-K1 (50-107) is uppercase |
| | LACI-K2 (121-178) is underscored |
| | LACI-K3 (211-270) is bold |
| BPTI | 1 2 3 4 5 |
| (SEQ ID | 1234567890123456789012345678901234567890123456789012345678 |
| NO: 2) | RPDFCLEPPYTGPCKARIIRYFYNAKAGLCQTFVYGGCRAKRNNFKSAEDCMRTCGGA |

The Kunitz domains above are referred to as LACI-K1 (residues 50 to 107), LACI-K2 (residues 121 to 178), and LACI-K3 (213 to 270). The cDNA sequence of 5 LACI is reported in Wun et al. (J. Biol. Chem. (1988) 263 (13): 6001-6004). Girard et al. (Nature (1989) 338:518-20) reports mutational studies in which the P1 residues of each of the three Kunitz domains were altered. LACI-K1 inhibits Factor VIIa (F.VIIa) when F.VIIa is complexed to tissue factor and LACI-K2 inhibits Factor Xa.

Proteins Containing Exemplary Kunitz Domains Include the Following, with SWISS-PROT Accession Numbers in Parentheses:

A4_HUMAN (P05067), A4_MACFA (P53601), A4_MACMU (P29216),
A4_MOUSE (P12023), A4_RAT (P08592), A4_SAISC (Q95241),
AMBP_PLEPL (P36992), APP2_HUMAN (Q06481), APP2_RAT (P15943),
AXP1_ANTAF (P81547), AXP2_ANTAF (P81548), BPT1_BOVIN (P00974),
BPT2_BOVIN (P04815), CA17_HUMAN (Q02388), CA36_CHICK (P15989),
CA36_HUMAN (P12111), CRPT_BOOMI (P81162), ELAC_MACEU (O62845),
ELAC_TRIVU (Q29143), EPPI_HUMAN (O95925), EPPI_MOUSE (Q9DA01),
HTIB_MANSE (P26227), IBP_CARCR (P00993), IBPC_BOVIN (P00976),
IBPI_TACTR (P16044), IBPS_BOVIN (P00975), ICS3_BOMMO (P07481),
IMAP_DROFU (P11424), IP52_ANESU (P10280), ISC1_BOMMO (P10831),
ISC2_BOMMO (P10832), ISH1_STOHE (P31713), ISH2_STOHE (P81129),
ISIK_HELPO (P00994), ISP2_GALME (P81906), IVB1_BUNFA (P25660),
IVB1_BUNMU (P00987), IVB1_VIPAA (P00991), IVB2_BUNMU (P00989),
IVB2_DABRU (P00990), IVB2_HEMHA (P00985), IVB2_NAJNI (P00986),
IVB3_VIPAA (P00992), IVBB_DENPO (P00983), IVBC_NAJNA (P19859),
IVBC_OPHHA (P82966), IVBE_DENPO (P00984), IVBI_DENAN (P00980),
IVBI_DENPO (P00979), IVBK_DENAN (P00982), IVBK_DENPO (P00981),
IVBT_ERIMA (P24541), IVBT_NAJNA (P20229), MCPI_MELCP (P82968),
SBPI_SARBU (P26228), SPT3_HUMAN (P49223), TKD1_BOVIN (Q28201),
TKD1_SHEEP (Q29428), TXCA_DENAN (P81658), UPTI_PIG (Q29100),
AMBP_BOVIN (P00978), AMBP_HUMAN (P02760), AMBP_MERUN (Q62577),
AMBP_MESAU (Q60559), AMBP_MOUSE (Q07456), AMBP_PIG (P04366),
AMBP_RAT (Q64240), IATR_HORSE (P04365), IATR_SHEEP (P13371),
SPT1_HUMAN (O43278), SPT1_MOUSE (Q9R097), SPT2_HUMAN (O43291),
SPT2_MOUSE (Q9WU03), TFP2_HUMAN (P48307), TFP2_MOUSE (O35536),
TFPI_HUMAN (P10646), TFPI_MACMU (Q28864), TFPI_MOUSE (O54819),
TFPI_RABIT (P19761), TFPI_RAT (Q02445), YN81_CAEEL (Q03610)

A variety of methods can be used to identify a Kunitz domain from a sequence database. For example, a known amino acid sequence of a Kunitz domain, a consensus sequence, or a motif (e.g., the ProSite Motif) can be searched against the GenBank sequence databases (National Center for Biotechnology Information, National Institutes of Health, Bethesda MD), e.g., using BLAST; against Pfam database of HMMs (Hidden Markov Models) (e.g., using default parameters for Pfam searching; against the SMART database; or against the ProDom database. For example, the Pfam Accession Number PF00014 of Pfam Release 9 provides numerous Kunitz domains and an HMM for identify Kunitz domains. A description of the Pfam database can be found in Sonhammer et al. Proteins (1997) 28(3): 405-420 and a detailed description of HMMs can be found, for example, in Gribskov et al. Meth. Enzymol. (1990) 183:146-159; Gribskov et al. Proc. Natl. Acad. Sci. USA (1987) 84:4355-4358; Krogh et al. J. Mol. Biol. (1994) 235:1501-1531; and Stultz et al. Protein Sci. (1993) 2:305-314. The SMART database (Simple Modular Architecture Research Tool, EMBL, Heidelberg, DE) of HMMs as described in Schultz et al. Proc. Natl. Acad. Sci. USA (1998) 95:5857 and Schultz et al. Nucl. Acids Res (2000) 28:231. The SMART database contains domains identified by profiling with the hidden Markov models of the HMMer2 search program (R. Durbin et al. (1998) Biological sequence analysis: probabilistic models of proteins and nucleic acids. Cambridge University Press). The database also is annotated and monitored. The ProDom protein domain database consists of an automatic compilation of homologous domains (Corpet et al. Nucl. Acids Res. (1999) 27:263-267). Current versions of ProDom are built using recursive PSI-BLAST searches (Altschul et al. Nucleic Acids Res. (1997) 25:3389-3402; Gouzy et al. Computers and Chemistry (1999) 23:333-340.) of the SWISS-PROT 38 and TREMBL protein databases. The database automatically generates a consensus sequence for each domain. Prosite lists the Kunitz domain as a motif and identifies proteins that include a Kunitz domain. See, e.g., Falquet et al. Nucleic Acids Res. (2002) 30:235-238.

Kunitz domains interact with target protease using, primarily, amino acids in two loop regions (“binding loops”). The first loop region is between about residues corresponding to amino acids 13-20 of BPTI. The second loop region is between about residues corresponding to amino acids 31-39 of BPTI. An exemplary library of Kunitz domains varies one or more amino acid positions in the first and/or second loop regions. Particularly useful positions to vary, when screening for Kunitz domains that interact with kallikrein or when selecting for improved affinity variants, include: positions 13, 15, 16, 17, 18, 19, 31, 32, 34, and 39 with respect to the sequence of BPTI. At least some of these positions are expected to be in close contact with the target protease. It is also useful to vary other positions, e.g., positions that are adjacent to the aforementioned positions in the three-dimensional structure.

The “framework region” of a Kunitz domain is defined as those residues that are a part of the Kunitz domain, but specifically excluding residues in the first and second binding loops regions, i.e., about residues corresponding to amino acids 13-20 of BPTI and 31-39 of BPTI. Conversely, residues that are not in the binding loop may tolerate a wider range of amino acid substitution (e.g., conservative and/or non-conservative substitutions).

In one embodiment, these Kunitz domains are variant forms of the looped structure including Kunitz domain 1 of human lipoprotein-associated coagulation inhibitor (LACI) protein. LACI contains three internal, well-defined, peptide loop structures that are paradigm Kunitz domains (Girard, T. et al., Nature (1989) 338:518- 520). Variants of Kunitz domain 1 of LACI described herein have been screened, isolated and bind kallikrein with enhanced affinity and specificity (see, for example, U.S. Pat. Nos. 5,795,865 and 6,057,287). These methods can also be applied to other Kunitz domain frameworks to obtain other Kunitz domains that interact with kallikrein, e.g., plasma kallikrein. Useful modulators of kallikrein function typically bind and/or inhibit kallikrein, as determined using kallikrein binding and inhibition assays.

In some aspects, the plasma kallikrein inhibitor binds to the active form of plasma kallikrein. In some embodiments, the plasma kallikrein inhibitor, binds to and inhibits plasma kallikrein, e.g., human plasma kallikrein and/or murine kallikrein. Exemplary polypeptide plasma kallikrein inhibitory agents are disclosed in U.S. Pat. Nos. 5,795,865, 5,994,125, 6,057,287, 6,333,402, 7,628,983, and 8,283,321, 7,064,107, 7,276,480, 7,851,442, 8,124,586, 7,811,991, and U.S. Publication No. 20110086801, the entire contents of each of which is incorporated herein by reference. In some embodiments, the plasma kallikrein inhibitor is an inhibitory polypeptide or peptide. In some embodiments, the inhibitory peptide is ecallantide (also referred to as DX-88 or KALBITOR®; SEQ ID NO: 3). In some embodiments, the plasma kallikrein inhibitor comprises or consists of an about 58-amino acid sequence of amino acids 3-60 of SEQ ID NO:3 or the DX-88 polypeptide having the 60-amino acid sequence of SEQ ID NO: 3:

(SEQ ID NO: 3)
Glu Ala Met His Ser Phe Cys Ala Phe Lys Ala Asp
Gly Pro Cys Arg Ala His Pro Arg Trp Phe Asn Ile
Phe Thr Arg Gln Cys Glu Phe Ile Tyr Gly Cys Glu
Gly Asn Gln Asn Arg Phe Glu Ser Leu Glu Cys Lys
Met Cys Thr Arg Asp.

The plasma kallikrein inhibitor can be a full-length antibody (e.g., an IgG (e.g., an IgG1, IgG2, IgG3, IgG4), IgM, IgA (e.g., IgA1, IgA2), IgD, and IgE)) or can include only an antigen-binding fragment (e.g., a Fab, F(ab′)2 or scFv fragment). The plasma kallikrein binding antibody can include two heavy chains and two light chains, or can be a single chain antibody. The plasma kallikrein inhibitor can be a recombinant protein such as humanized, CDR grafted, chimeric, deimmunized, or in vitro generated antibodies, and may optionally include constant regions derived from human germline immunoglobulin sequences. In one embodiment, the plasma kallikrein inhibitor is a monoclonal antibody.

Exemplary plasma kallikrein binding proteins are disclosed in U.S. Publication No. 2012/0201756, the entire contents of which are incorporated herein by reference. In some embodiments, the plasma kallikrein binding protein is an antibody (e.g., a human antibody) having the light and/or heavy chains of antibodies selected from the group consisting of M162-A04, M160-G12, M142-H08, X63-G06, X101-A01 (also referred to as DX-2922), X81-B01, X67-D03, X67-G04, X81-B01, X67-D03, X67-G04, X115-B07, X115-D05, X115-E09, X115-H06, X115-A03, X115-D01, X115-F02, X124-G01 (also referred to herein as DX-2930 or lanadelumab), X115-G04, M29-D09, M145-D11, M06-D09 and M35-G04. In some embodiments, the plasma kallikrein binding protein competes with or binds the same epitope as M162-A04, M160-G12, M142-H08, X63-G06, X101-A01 (also referred to herein as DX-2922), X81-B01, X67-D03, X67-G04, X81-B01, X67-D03, X67-G04, X115-B07, X115-D05, X115-E09, X115-H06, X115-A03, X115-D01, X115-F02, X124-G01, X115-G04, M29-D09, M145-D11, M06-D09 and M35-G04. In some embodiments, the plasma kallikrein binding protein is lanadelumab. See US Publication No. 2011/0200611 and US Publication No. 2012/0201756, which are incorporated by reference herein.

An example of a plasma kallikrein inhibitory antibody is lanadelumab (TAKHZYROR, which may be also referred to as TAK-743, SHP943, or DX-2930). The amino acid sequences of the heavy chain and light chain variable regions of lanadelumab are provided below with the CDR regions identified in boldface and underlined.

Lanadelumab heavy chain variable region sequence
(SEQ ID NO: 4)
EVQLLESGGG LVQPGGSLRL SCAASGFTFS HYIMMWVRQA
PGKGLEWVSG IYSSGGITVY ADSVKGRFTI SRDNSKNTLY
LQMNSLRAED TAVYYCAYRR IGVPRRDEFD IWGQGTMVTV
SS
Lanadelumab light chain variable region sequence
(SEQ ID NO: 5)
DIQMTQSPS TLSASVGDRV TITCRASQSI SSWLAWYQQK
PGKAPKLLIY KASTLESGVP SRFSGSGSGT EFTLTISSLQ
PDDFATYYCQ QYNTYWTFGQ GTKVEI

The heavy and light chain constant and variable sequences for lanadelumab are provided below, with signal sequences in italics. The CDRs are boldfaced and underlined.

Lanadelumab Heavy Chain Amino Acid Sequence
(451 amino acids, 49439.02 Da)
(SEQ ID NO: 6)
MGWSCILFLVATATGAHSEVQLLESGGGLVQPGGSLRLSCAASGFT
FSHYIMMWVRQAPGKGLEWVSGIYSSGGITVYADSVKGRFTISRDN
SKNTLYLQMNSLRAEDTAVYYCAYRRIGVPRRDEFDIWGQGTMVTV
SSASTKGPSVFPLAPSSKSTSGGTAALGCLVKDYFPEPVTVSWNSG
ALTSGVHTFPAVLQSSGLYSLSSVVTVPSSSLGTQTYICNVNHKPS
NTKVDKRVEPKSCDKTHTCPPCPAPELLGGPSVFLFPPKPKDTLMI
SRTPEVTCVVVDVSHEDPEVKFNWYVDGVEVHNAKTKPREEQYNST
YRVVSVLTVLHQDWLNGKEYKCKVSNKALPAPIEKTISKAKGQPRE
PQVYTLPPSREEMTKNQVSLTCLVKGFYPSDIAVEWESNGQPENNY
KTTPPVLDSDGSFFLYSKLTVDKSRWQQGNVFSCSVMHEALHNHYT
QKSLSLSPG
Lanadelumab Light Chain Amino Acid Sequence
(213 amino acids, 23419.08 Da)
(SEQ ID NO: 7)
MGWSCILFLVATATGAHSDIQMTQSPSTLSASVGDRVTITCRASQS
ISSWLAWYQQKPGKAPKLLIYKASTLESGVPSRFSGSGSGTEFTLT
ISSLQPDDFATYYCQQYNTYWTFGQGTKVEIKRTVAAPSVFIFPPS
DEQLKSGTASVVCLLNNFYPREAKVQWKVDNALQSGNSQESVTEQD
SKDSTYSLSSTLTLSKADYEKHKVYACEVTHQGLSSPVTKSFNRGE
C

TABLE 4
CDRs for lanadelumab

| CDR | Amino acid sequence |
|---|---|
| Heavy chain CDR1 | HYIMM (SEQ ID NO: 8) |
| Heavy chain CDR2 | GIYSSGGITVYADSVKG (SEQ ID NO: 9) |
| Heavy chain CDR3 | RRIGVPRRDEFDI (SEQ ID NO: 10) |
| Light chain CDR1 | RASQSISSWLA (SEQ ID NO: 11) |
| Light chain CDR2 | KASTLES (SEQ ID NO: 12) |
| Light chain CDR3 | QQYNTYWT (SEQ ID NO: 13) |

In some embodiments, a plasma kallikrein inhibitor can have at least 85%, 90%, 91%, 92%, 93%, 94%, 95%, 96%, 97%, 98%, 99% or higher sequence identity to a plasma kallikrein inhibitor described herein. In some embodiments, a plasma kallikrein inhibitor can have at least 85%, 90%, 91%, 92%, 93%, 94%, 95%, 96%, 97%, 98%, 99% or higher sequence identity in the HC and/or LC framework regions (e.g., HC and/or LC FR 1, 2, 3, and/or 4) to a plasma kallikrein inhibitor described herein. In some embodiments, a plasma kallikrein inhibitor can have at least 85%, 90%, 91%, 92%, 93%, 94%, 95%, 96%, 97%, 98%, 99% or higher sequence identity in the HC and/or LC CDRs (e.g., HC and/or LC CDR1, 2, and/or 3) to a plasma kallikrein inhibitor described herein. In some embodiments, a plasma kallikrein inhibitor can have at least 85%, 90%, 91%, 92%, 93%, 94%, 95%, 96%, 97%, 98%, 99% or higher sequence identity in the constant region (e.g., CH1, CH2, CH3, and/or CL1) to a plasma kallikrein inhibitor described herein.

In some aspects, a small molecule binds and inhibits the active form of plasma kallikrein.

### Bradykinin B2 Receptor Inhibitors

In some embodiments, a bradykinin B2 receptor inhibitor (e.g., antagonist) is administered to a subject. Exemplary bradykinin B2 receptor antagonists include icatibant (FIRAZYR®), which is a peptidomimetic drug containing 10 amino acids which block binding of native bradykinin to the bradykinin B2 receptor.

### C1-INH Replacement Agents

In some embodiment, a C1 esterase inhibitor (C1-INH), such as a replacement C1-INH agent is administered to a subject. Exemplary C1-INH replacement agents are publicly available and include, for example, human plasma-derived C1-INH, e.g. BERINERT® and CINRYZE®.

### Kits and Detecting Devices for Measuring Protein Biomarker Sets

The present disclosure also provides kits and detecting devices for use in measuring the level of a biomarker set as described herein. Such a kit or detecting device can comprise binding agents that specifically bind to protein biomarkers, such as those listed in Table 1. For example, such a kit or detecting device may comprise at least two binding agents that are specific to two different protein biomarkers selected from Table 1. In some instances, the kit or detecting device comprises binding agents specific to all members of the protein biomarker set described herein.

In some embodiments, one or more of the binding agents is an antibody that specifically binds to a protein of the biomarker set. In some embodiments, the one or more binding agents is an aptamer, such as a peptide aptamer or oligonucleotide aptamer, that specifically binds to a protein of the biomarker set.

In some embodiments, the kits further comprise a detection agent (e.g., an antibody binding to the binding agent) for detecting binding of the agent to the protein(s) of the biomarker set. The detection agent can be conjugated to a label. In some embodiments, the detection agent is an antibody that specifically binds to at least one of the binding agents. In some embodiments, the binding agent comprises a tag that can be identified and, directly or indirectly, bound by a detection agent.

In some embodiments, the kits may further comprise a support member, for example for performing any of the methods described herein. In some embodiments, the support member is a membrane, such as a nitrocellulose membrane, a polyvinylidene fluoride (PVDF) membrane, or a cellulose acetate membrane. In some examples, the immunoassay may be in a Western blot assay format or a lateral flow assay format.

In some embodiments, the support member is a multi-well plate, such as an ELISA plate. In some embodiments, the immunoassays described herein can be carried out on high throughput platforms. In some embodiments, multi-well plates, e.g., 24-, 48-, 96-, 384- or greater well plates, may be used for high throughput immunoassays. Individual immunoassays can be carried out in each well in parallel. Therefore, it is generally desirable to use a plate reader to measure multiple wells in parallel to increase assay throughput. In some embodiments, plate readers that are capable of imaging multi-wells (e.g., 4, 16, 24, 48, 96, 384, or greater wells) in parallel can be used for this platform. For example, a commercially available plate reader (e.g., the plate::vision system available from Perkin Elmer, Waltham, MA) may be used. This plate reader is capable of kinetic-based fluorescence analysis. The plate::vision system has high collection efficiency optics and has special optics designed for the analysis of 96 wells in parallel. Additional suitable parallel plate readers include but are not limited to the SAFIRE (Tecan, San Jose, CA), the FLIPRTETRA® (Molecular Devices, Union City, CA), the FDSS7000 (Hamamatsu, Bridgewater, NJ), and the CellLux (Perkin Elmer, Waltham, MA).

In the kit or detecting device, one or more of the binding agents may be immobilized on a support member, e.g., a membrane, a bead, a slide, or a multi-well plate. Selection of an appropriate support member for the immunoassay will depend on various factor such as the number of samples and method of detecting the signal released from label conjugated to the second agent.

The kit can also comprise one or more buffers as described herein but not limited to a coating buffer, a blocking buffer, a wash buffer, and/or a stopping buffer.

In some embodiments, the kit can comprise instructions for use in accordance with any of the methods described herein. The included instructions can comprise a description of how to use the components contained in the kit for measuring the level of proteins of a biomarker set in a biological sample collected from a subject, such as a human patient.

The instructions relating to the use of the kit generally include information as to the amount of each component and suitable conditions for performing the assay methods described herein. The components in the kits may be in unit doses, bulk packages (e.g., multi-dose packages), or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the kit is used for evaluating the level of proteins of a biomarker set. Instructions may be provided for practicing any of the methods described herein.

The kits of this present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as interpretive information, such as a control and/or standard or reference sample. Normally, the kit comprises a container and a label or package insert(s) on or associated with the container. In some embodiments, the present disclosure provides articles of manufacture comprising contents of the kits described above.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Identification of Proteins Differentially Present in Samples from HAE Patients as Compared to Healthy Individuals

Despite the clinically validated role of the kallikrein-kinin system (KKS) in hereditary angioedema (HAE) C1 esterase inhibitor (C1-INH) pathophysiology (HAE-C1-INH), novel disease state biomarkers useful for diagnosing and evaluating HAE patients remain to be identified. Such biomarkers may be useful for further elucidating the disease biology of HAE and other plasma kallikrein (pKal)-mediated diseases, for the development of improved diagnostic assays, as well as for the identification of additional indications beyond HAE that may be susceptible to treatment with established therapeutics, such as lanadelumab. To identify previously unrecognized HAE biomarkers, the plasma proteome of HAE subjects was analyzed before (baseline) and after 6 months (26 weeks) of lanadelumab treatment and compared with analyte levels present in plasma samples from healthy control subjects (subjects not having HAE).

Positive determination of HAE in patients was performed by collecting plasma samples from subjects and measuring plasma levels of complement component 4 (C4) and functional C1-INH via ELISA and chromogenic assays known in the art, respectively. Plasma proteomic analyses were performed by using a multiplex approach capable of assessing the relative levels of 7,113 proteins using DNA aptamers specific for each assessed protein (SOMALogic, Denver, CO, USA), and comparing the mean levels measured in plasma samples obtained from healthy control subjects (n=30), plasma samples obtained from HAE patients (HAE C1-INH) prior to lanadelumab treatment (baseline, n=125), and plasma samples obtained from HAE patients following lanadelumab treatment of 300 mg lanadelumab every two weeks (week 26, post treatment, n=114). Of the 114 HAE-C1-INH subjects for which baseline/pre-dose plasma was available, matched post-dose samples were available for 112 subjects.

### Plasma Collection

Citrated plasma samples from healthy controls were age and gender matched (n=30; BioIVT, Westbury, NY, USA). Citrated plasma samples from HAE-C1-INH subjects prior to lanadelumab treatment (n=125) were collected as part of a phase III study (HELP study) and non-rollovers from an open label expansion study (the open label extension portion of the HELP study) (National Institutes of Health identifiers NCT02586805 and NCT02741596, respectively). Citrated plasma samples from HAE-C1-INH subjects after 26 weeks of lanadelumab treatment (300 mg lanadelumab administered every 2 weeks; n=114) were collected, with patient-matched pre-treatment and post-treatment samples available for 112 subjects. Plasma (sodium citrate) was collected from age and gender matched healthy controls (n=30) by BioIVT (Westbury, NY). To minimize ex vivo activation of the contact pathway system during blood collection, plasma was collected from subjects with HAE-C1-INH and healthy controls by means of a clean venipuncture with a butterfly needle/catheter kit (BD Biosciences) and removal of the tourniquet upon blood flow to decrease stasis. The first tube of blood was discarded, and blood was collected into polypropylene evacuated tubes containing 3.8% sodium citrate (BD Biosciences, San Jose, CA, USA). Blood samples were centrifuged within 1 hour, and plasma was aliquoted and stored at −80° C. until processing.

### ELISA

For validation of differentially expressed HAE biomarkers, protein levels were assessed by ELISA. A2M was measured in plasma samples using a commercially available ELISA kit (Raybiotech, cat. #ELH-A2M). APOB was measured in plasma samples using a commercially available ELISA kit (Raybiotech, cat. #ELH-ApoB). IL21 was measured in plasma samples using a commercially available ELISA kit (Raybiotech, cat. #ELH-IL21).

### Results

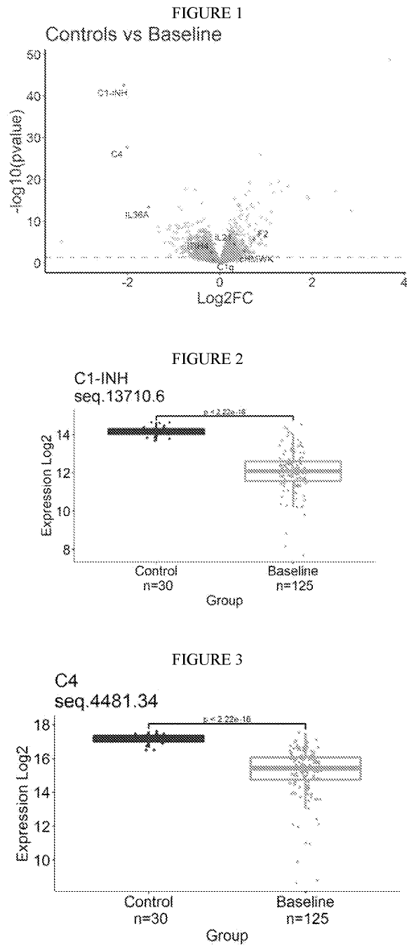

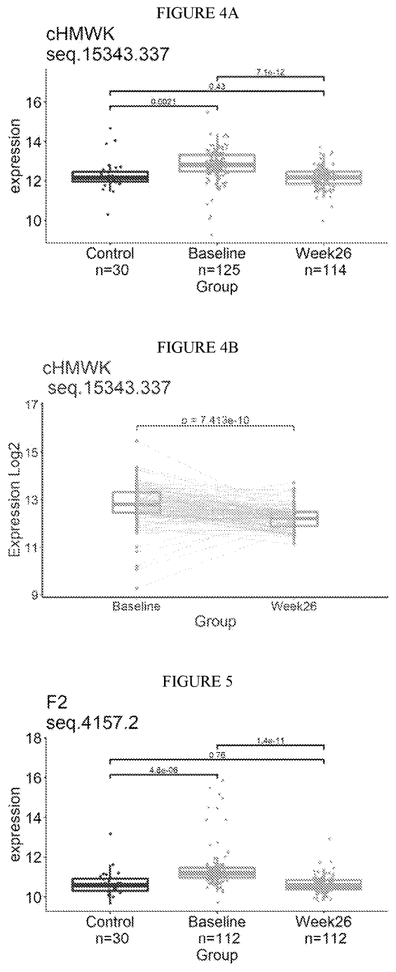

Of the 7,113 proteins assessed, 1041 proteins were determined to be differentially at statistically significant levels in samples obtained from HAE patients at baseline, as compared to healthy controls (FIG. 1). As validation of this approach, various biomarkers that are previously known to be differentially present during HAE were identified within this set. For example, C1-INH and C4 each of which are biomarkers utilized in laboratory diagnosis of HAE-C1-INH (Grumach et al. (2021) Front Immunol, 12:785736), were found to be reduced in HAE baseline samples (FIG. 2 and FIG. 3, respectively). Cleaved high molecular weight kininogen (cHMWK), which is known to be elevated during HAE-C1-INH and reduced following lanadelumab treatment, was also observed in the analysis (FIGS. 4A and 4B). See, e.g., Suffritti et al. (2014) Clin Exp Allergy, 44:1503-14; Hofman et al. (2017) J Allergy Clin Immunol. 140:1700-1703; and Banerji et al. (2017) N Engl J Med., 376:717-728. Several additional proteins previously reported to be differentially present during HAE-C1-INH disease state were also observed in the analysis, including thrombin, ITH4, IL-36A, and IL-21 (see, FIGS. 5-8). See, e.g., van Geffen et al. (2012) Clin Exper Immunol, 167:472-478; Larrauri et al. (2020) Mol Immunol, 119:27-34; Sexton et al. (2017) Allergy, 0207; and Arcoleo et al. (2018) Clin Exp Med, 18:355-361.

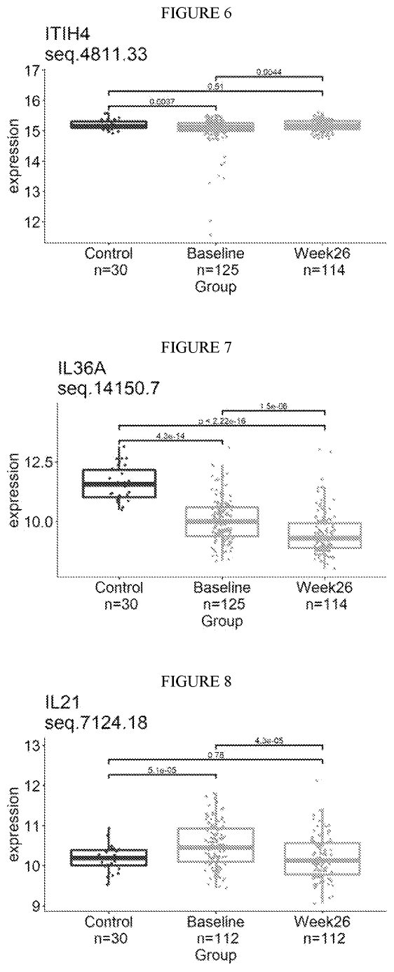

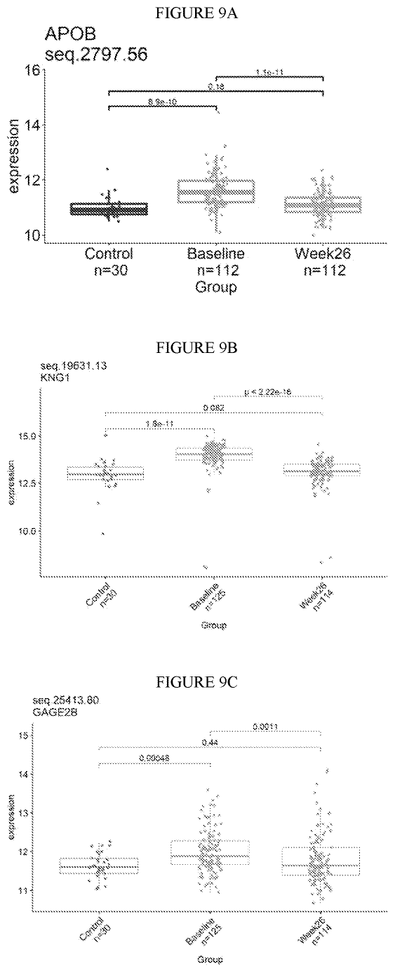

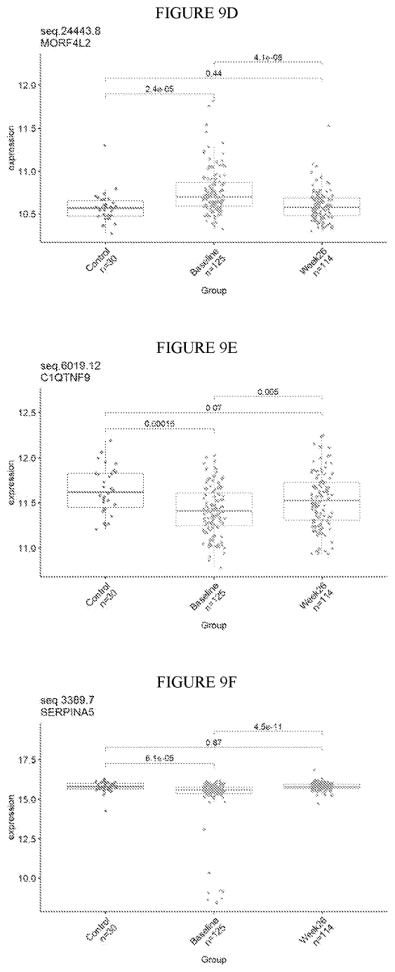

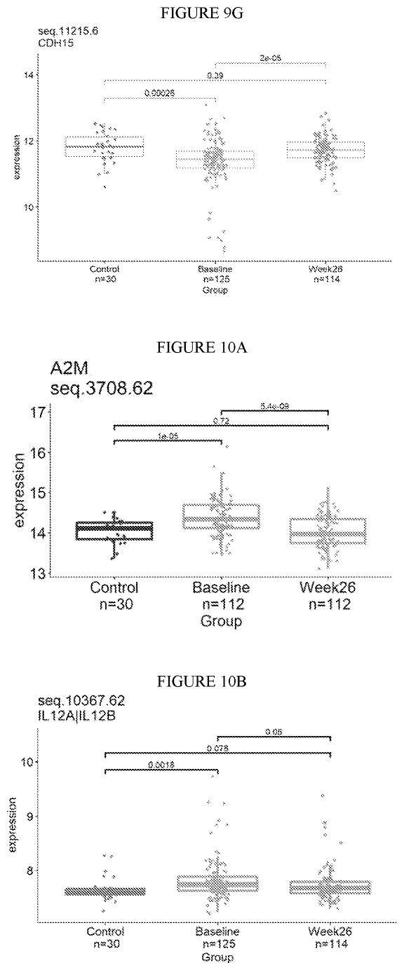

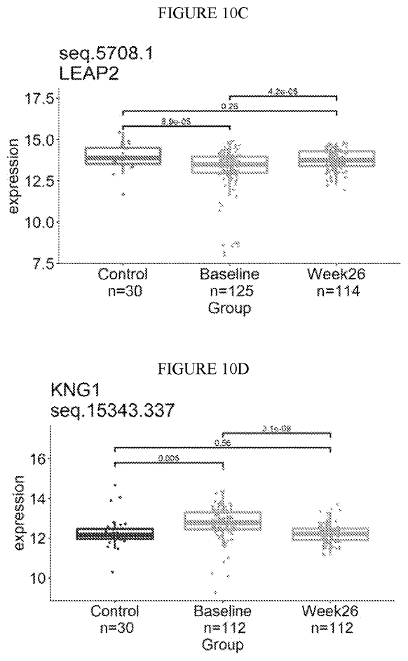

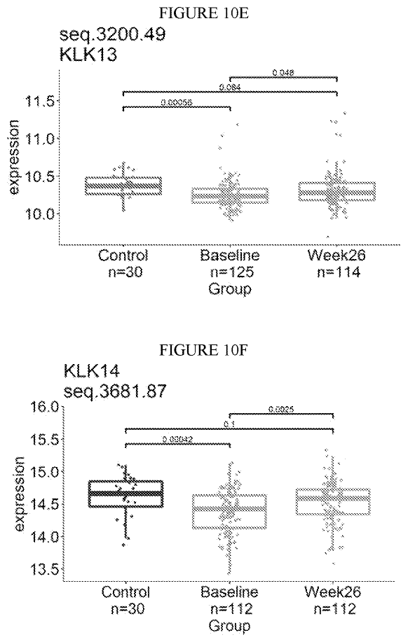

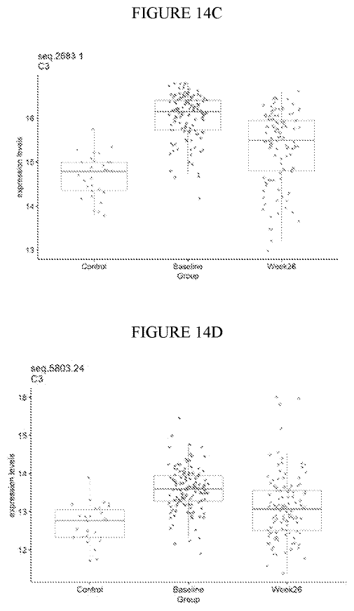

Statistical analysis was performed to identify proteins that differed between samples from HAE-C1-INH patients as compared to healthy subjects, as well as to identify proteins that did not differ after lanadelumab treatment (i.e., returned to control levels). As shown in Table 1, the levels of 120 proteins were further found to differ significantly in HAE-C1-INH plasma at baseline as compared to healthy controls but did not differ significantly (normalized) after treatment with lanadelumab for 26 weeks. These proteins include several biomarkers previously known to differ significantly during HAE-C1-INH, namely cleaved HMWK (cHMWK; FIG. 4A), thrombin (F2; FIG. 5), inter-α-trypsin inhibitor heavy chain H4 (ITH4; FIG. 6), IL-21 (FIG. 8), as well as numerous proteins not previously recognized to be differentially present during HAE-C1-INH and/or following treatment with a plasma kallikrein inhibitor such as lanadelumab. Additional proteins identified, include α-macroglobulin (A2M; FIG. 10A), kininogen (KNG1; FIGS. 9B, 10D, and 13A-13H) complement component C3 (C3; FIGS. 14A-14D), and plasminogen (PLG; data not shown).

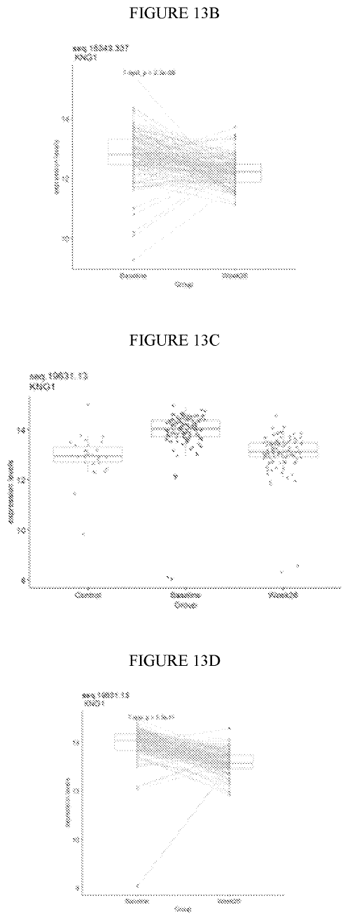

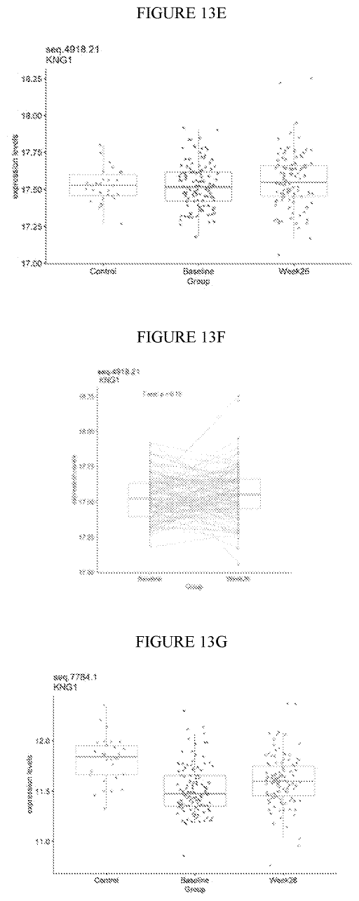

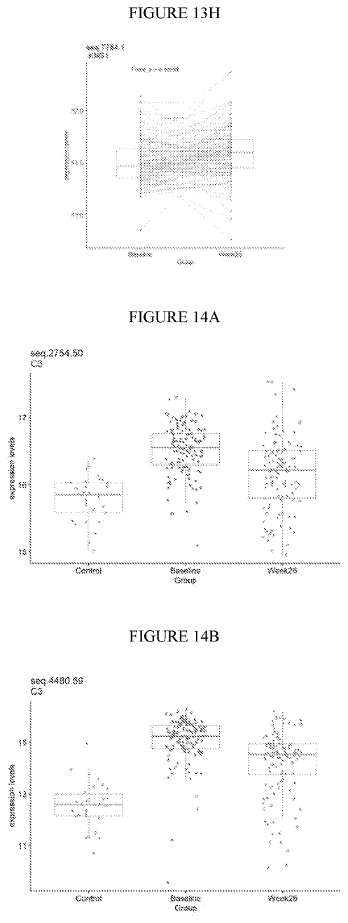

As an example of how these 120 proteins were identified, DNA aptamers that specifically bind cHMWK (ID 15343-337 and ID 19631-13; Table 1; FIGS. 13A-13D) show that cHMWK levels were elevated in HAE-C1-INH baseline plasma but were no longer different from healthy control plasma levels after HAE-C1-INH subjects were treated with lanadelumab for 26 weeks. A DNA aptamer raised against intact HMWK with no other specificity information provided by SOMALogic showed similar relative expression levels in plasma samples of healthy controls, HAE-C1-INH patients at baseline, and HAE-C1-INH patients after 26 weeks of lanadelumab treatment (ID 4918-21; Table 1; FIGS. 13E-13F). A second DNA aptamer selective for intact HMWK, which was observed to bind cHMWK and low molecular weight kininogen with at least 10-fold lower affinity when compared to intact HMWK, shows decreased expression level in HAE-C1-INH baseline plasma relative to healthy controls and increased expression level in HAE-C1-INH subjects treated with lanadelumab for 26 weeks (ID 7784-1; Table 1; FIGS. 13G-13H).

Complement C3 (C3) is another example among the list of 120 proteins of a potential biomarkers identified by proteomics using HELP plasma samples. Increased expression levels of C3 and C3 fragments were observed in baseline plasma samples of HAE-C1-INH subjects as compared to healthy controls (FIGS. 14A-14D). C3b expression levels (ID 4480-59; Table 1; FIG. 14B) were elevated in baseline plasma samples of HAE-C1-INH subjects and reduced after 26 weeks of receiving 300 mg of lanadelumab every 4 weeks.

These proteins also include many biomarkers not previously recognized to differ significantly during HAE-C1-INH or to normalize to levels comparable to that of a healthy subject after treatment with a plasma kallikrein inhibitor such as lanadelumab. Examples of such proteins include apolipoprotein B (APOB; FIG. 9A), G antigen 2 (GAGE2B; FIG. 9C), mortality factor 4-like protein 2 (MORF4L2; FIG. 9D), complement C1q and tumor necrosis factor-related protein 9A (C1QTNF9; FIG. 9E), plasma serine protease inhibitor (SERPINA5; FIG. 9F), and cadherin-15:cytoplasmic domain (CDH15; FIG. 9G), alpha-2-macroglobulin (A2M; FIG. 10A), interleukin-12 (IL12A-IL12B; FIG. 10B), and liver-expressed antimicrobial peptide 2 (LEAP2; FIG. 10C) (FIGS. 9A-9G, 10A-10C; Table 1). Gene names, UniProt identifiers, and identifiers for SomaScan® aptamers utilized for analyzing expression levels for each protein are also provided in Table 1.

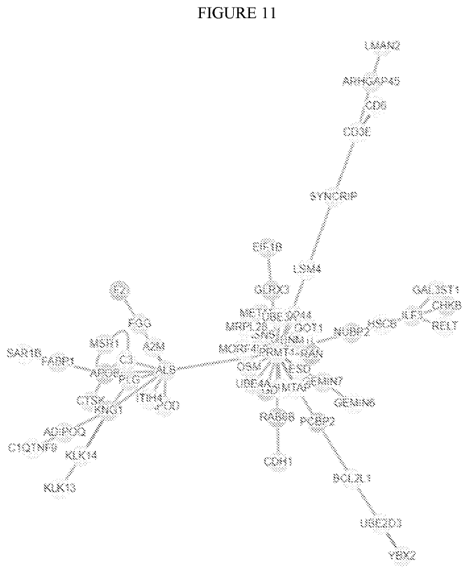

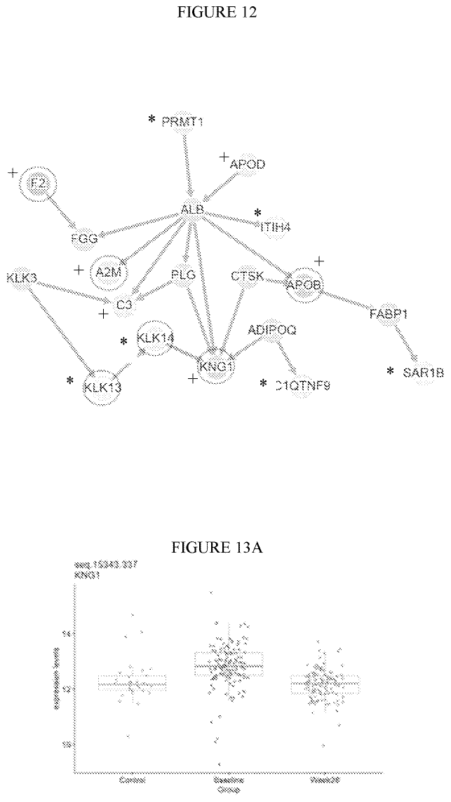

To determine if these 120 proteins represented components of pathways functionally related to the pathophysiology of HAE, a pathway enrichment analysis was performed using Metabase process networks (bio.tools/metabase; Bolser et al., (2012) Nucleic Acids Res, 40:D1250-D1254) and a Known Knowledge Network approach referred to as CASnet (consistently active subnetworks of known protein-protein interactions). Proteins that are functionally involved in the kallikrein-kinin system were among the most significantly enriched. However, proteins involved in coagulation, cell adhesion, cytokines, cardiovascular proteins, and proteolysis (including proteases and proteolysis inhibitors) pathways, among others, were also found to be significantly enriched, suggesting that these pathways are also dysregulated during HAE-C1-INH and become normalized following lanadelumab treatment. Summarized results of Metabase pathway enrichment analysis are provided in Tables 3 and 5 and FIGS. 11 and 12.

CASnet incorporates the directionality of the change in protein level, where proteins in red were elevated, while those in blue were decreased when HAE baseline was compared to healthy control plasma (FIG. 12). The indicated proteins were added to the active subnetwork via known pathway associations. Proteins that were identified by CASnet analyses as potential disease state biomarkers impacted by 26 weeks of lanadelumab treatment include proteases and protease inhibitors (e.g., thrombin, tissue kallikrein 14, Inter-alpha trypsin inhibitor heavy chain 4, α2-macrogrobulin), apolipoproteins, and complement system proteins.

TABLE 3
Metabase pathway analysis of 120 proteins differentially expressed in plasma
samples from HAE patients prior to but not after lanadelumab treatment

| Pathway | r | R | n | N | Z score | p value | q value |
|---|---|---|---|---|---|---|---|
| Kallikrein-Kinin System | 13 | 68 | 187 | 7113 | 8.538585 | 0 | 3e−06 |
| Blood coagulation | 5 | 68 | 93 | 7113 | 4.409437 | 0.00186 | 0.138223 |
| Cell adhesion_Amyloid proteins | 7 | 68 | 197 | 7113 | 3.799111 | 0.00260 | 0.138223 |
| Cell adhesion Platelet- | 6 | 68 | 174 | 7113 | 3.420398 | 0.00613 | 0.243986 |
| endothelium-leucocyte | | | | | | | |
| interactions | | | | | | | |
| Proteolysis_Connective tissue | 4 | 68 | 118 | 7113 | 2.73963 | 0.02597 | 0.707345 |
| degradation | | | | | | | |

Pathway enrichment analysis was performed using hypergeometric testing, where “r” represents number of proteins belonging to a given pathway, “R” represents total number of proteins tested, “n” represents number of proteins in a given pathway, and “N” represents total number of proteins in all pathways.

TABLE 5
Pathway analysis comparing HAE-C1-INH baseline to healthy control plasma

| Pathway (# proteins in pathway) | # Proteins* | | P-values |
|---|---|---|---|
| Blood coagulation (33) | 14 | (42%) | 1.22E−04 |
| Bradykinin/Kallidin maturation (35) | 11 | (31%) | 3.51E−03 |
| Role of adhesion of SCLC cells in tumor progression (42) | 10 | (24%) | 2.97E−02 |
| Role of proteases in hematopoietic stem cell mobilization (18) | 8 | (44%) | 3.65E−03 |
| Induction of the antigen presentation machinery by IFN-gamma | 11 | (16%) | 4.76E−02 |
| (68) | | | |
| Release of pro-inflammatory mediators and elastolytic enzymes by | 8 | (27%) | 2.47E−03 |
| alveolar macrophages in COPD (30) | | | |
| Cell-matrix glycoconjugates (39) | 10 | (26%) | 2.47E−03 |
| ECM remodeling (66) | 13 | (20%) | 3.33E−03 |
| Role of TGF-beta 1 in fibrosis development after myocardial | 7 | (19%) | 3.65E−03 |
| infarction (37) | | | |
| Resolution of inflammation in healing myocardial infarction (29) | 10 | (34%) | 4.52E−02 |

*Number of proteins identified out of the 1041 proteins identified in each pathway. The value in parenthesis is the percent of total proteins in the pathway.

The proteomic analysis identified 120 proteins that were present at levels that differed between samples from patients with HAE and healthy individuals (control) but normalized after 6 months (26 weeks) of lanadelumab treatment.

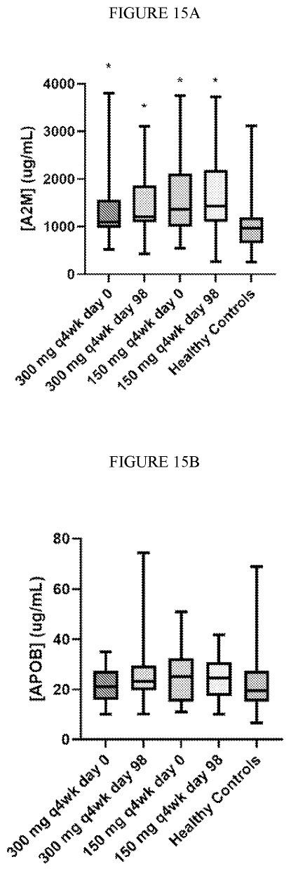

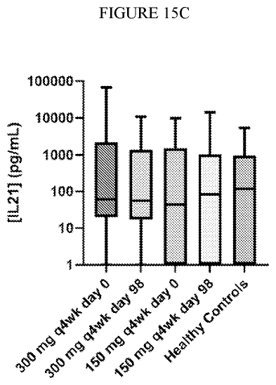

For three proteins (A2M, APOB, and IL21) identified herein, commercially available ELISA kits (Raybiotech, cat. #ELH-A2M, Raybiotech, cat. #ELH-IL21, and Raybiotech, cat. #ELH-ApoB) were used as orthogonal assays to compare levels in a different group of healthy controls (n=50) and HAE-C1-INH subjects from the HELP study (NCT02586805) baseline (prior to receiving lanadelumab) and after receiving either 150 mg or 300 mg lanadelumab once per month at day 98 after the first dose (FIG. 15A-15D).

The results of the analyses are shown in FIG. 15A (A2M), FIG. 15B (APOB) and FIG. 15C (IL21). Plasma concentrations of A2M in HAE-C1-INH subjects were significantly different than that of healthy controls (Kruskal-Wallis test, p<0.05) but did not differ between day 0 and day 98 following lanadelumab treatment, in contrast to the SomaScan measurement of A2M. Also in contrast to the SomaScan measurements, the concentrations of APOB and IL21 measured by ELISA were not significantly different from healthy controls or between day 0 and day 98 for each dose of lanadelumab.

Further studies could elucidate whether the Somamer against A2M also binds the A2M-PKa covalent complex to determine whether the observed increase in A2M is due to the increase in either free A2M protein or complexes with proteases. A significant increase in plasma A2M concentrations in HAE-C1-INH pre-dose plasma over that of healthy control plasma also was observed. However, by ELISA the concentration of A2M in HAE-C1-INH pre-dose plasma did not decrease with lanadelumab treatment. The absence of an observed decrease in A2M plasma levels by ELISA due to lanadelumab treatment may be attributed to several considerations: 1) results of different immunoassays can disagree with each other, so disagreement between the ELISA and SomaScan results does not definitively imply which is superior; 2) differences in assay characteristics (e.g. target immobilization and washing conditions) can influence comparisons across platforms; 3) assay specificity for different forms of the target (e.g. free A2M or protease-A2M covalent complexes); 4) SomaScan analyses were performed on plasma from subjects that received a higher dose of lanadelumab (300 mg q2wk) for a longer period (6 months) than for the plasma samples used in the ELISA analyses (300 mg or 150 mg q4wk for 98 days), which could suggest that higher and longer exposure of lanadelumab is required to reduce A2M. The same plasma samples used to measure A2M by ELISA were also used for the measurement of IL21 and APOB. However, the increased plasma levels from HAE-C1-INH subjects were not observed by ELISA, which could be attributed to the reasons described above for A2M.

Any of the proteins identified herein, may be used as a biomarker (individually or in combination as a biomarker set) for identifying diseases associated with the contact activation system, for example, in methods for identifying patients who are at risk of a disease associated with the contact activation system (e.g., HAE), selecting a candidate for treatment, monitoring disease progression or disease state, assessing the efficacy of a treatment against a disease, determining a course of treatment, identifying whether a disease or disorder is associated with the contact activation system, and/or for research purposes, including, e.g., studying the mechanism of a disease, which may be relied upon for the development of new therapies.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS AND SCOPE

Those skilled in the art will recognize, or be able to ascertain using no more than routine experimentation, many equivalents to the specific embodiments of the present disclosure described herein. The scope of the present disclosure is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method for determining if a disorder is susceptible to treatment with a plasma kallikrein (pKal) inhibitor, comprising
measuring the level of a biomarker set, which comprises at least one protein selected from Table 1, in a biological sample of a subject having the disorder, and
identifying the disorder as being susceptible to treatment with a pKal inhibitor if the level of the biomarker set deviates from a reference value.

2. The method of claim 1, further comprising administering the pKal inhibitor to the subject if the disorder is identified as susceptible to treatment with the pKal inhibitor.

3. A method for identifying a subject as a candidate for treatment with a plasma kallikrein (pKal) inhibitor, comprising
providing a biological sample from the subject having, suspected of having, or at risk of having a disorder, and
measuring the level of a biomarker set, which comprises at least one protein selected from Table 1, in the biological sample,
wherein if the level of the biomarker set in the biological sample deviates from a reference value, the subject is identified as a candidate for treatment with the pKal inhibitor.

4. The method of claim 3, further comprising administering the pKal inhibitor to the subject identified as a candidate for treatment.

5. A method for identifying a subject as having or at risk of having a disorder, comprising
providing a biological sample from the subject, and
measuring the level of a biomarker set, which comprises at least one protein selected from Table 1, in the biological sample,
wherein if the level of the biomarker set in the biological sample deviates from the level of the biomarker set in a control sample, the subject is identified as having or at risk of the disorder.

6. The method of claim 5, further comprising administering to the subject an effective amount of a plasma kallikrein (pKal) inhibitor, if the subject is identified as having or at risk of having the disorder.

7. A method for treating a disorder in a subject, comprising
administering to the subject an effective amount of a plasma kallikrein (pKal) inhibitor,
wherein the subject has a level of a biomarker set that deviates from the level of the biomarker set in a control sample,
wherein the biomarker set comprises at least one protein selected from Table 1.

8. The method of claim 7, wherein the disorder is not hereditary angioedema (HAE).

9. The method of any one of claims 1-8, wherein the pKal inhibitor is lanadelumab.

10. The method of any one of claims 1-9, wherein the biomarker set consists of 2-10 proteins selected from Table 1.

11. The method of any one of claims 1-10, wherein the biological sample is a serum sample or a plasma sample.

12. The method of any one of claims 1-11, wherein the disorder is a disease associated with the contact activation system.

13. The method of claim any one of claim 1-7 or 9-12, wherein the disorder is hereditary angioedema (HAE).

14. The method of claim any one of claim 1-7 or 9-13, wherein the disorder is type I HAE or type II HAE.

15. The method of any one of claims 1-14, wherein the at least one protein is a kallikrein-kinin system protein selected from the group consisting of kallikrein-13 (KLK13), kallikrein-14 (KLK14), 2-chain high molecular weight kininogen (KNG1), and kininogen.

16. The method of any one of claims 1-15, wherein the at least one protein is a blood coagulation protein selected from the group consisting of alpha-2-macroglobulin (A2M), complement C1q and tumor necrosis factor-related protein 9A (C1QTNF9), thrombin, and plasma serine protease inhibitor (SERPINA5).

17. The method of any one of claims 1-16, wherein the at least one protein is a cell adhesion protein selected from the group consisting of cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), and protocadherin gamma-C3 (PCDHGC3).

18. The method of any one of claims 1-17, wherein the at least one protein is a proteolysis-related protein selected from the group consisting of proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), and E3 ubiquitin-protein ligase ZNRF3 (ZNRF3).

19. The method of any one of claims 1-18, wherein the at least one protein is a complement activation protein selected from the group consisting of complement C1q and tumor necrosis factor-related protein 9A (C1QTNF9).

20. The method of any one of claims 1-19, wherein the at least one protein is selected from the group consisting of thrombin (F2), tissue kallikrein 14 (KLK4), tissue kallikrein 13 (KLK13), Inter-α-trypsin inhibitor heavy chain H4 (ITIH4), α-macroglobulin, Apolipoprotein B (APOB), interleukin-21 (IL-21), complement component C3 (C3), kininogen (KNG1), Protein arginine N-methyltransferase 1 (PRMT1), and Complement C1q and tumor necrosis factor-related protein 9A (C1QTNF9).

21. The method of any one of claims 1-20, wherein the biological sample is provided in an evacuated blood collection tube, which comprises one or more protease inhibitors.

22. The method of any one of claims 1-21, wherein the level of the biomarker set is measured by enzyme-linked immunosorbent assay (ELISA), an immunoblotting assay, or a lateral flow assay.

23. The method of any one of claims 1-22, wherein the subject is a human patient.

24. A composition for use in treating a subject having a disorder, the composition comprising a plasma kallikrein (pKal) inhibitor,
wherein the subject has a level of a biomarker set that deviates from the level of the biomarker set in a control sample,
wherein the biomarker set comprises at least one protein selected from Table 1.

25. The composition of claim 24, wherein the pKal inhibitor is lanadelumab.

26. The composition of claim 24 or 25, wherein the biomarker set consists of 2-10 proteins selected from Table 1.

27. The composition of any one of claims 24-26, wherein the disorder is a disease associated with the contact activation system.

28. The composition of any one of claims 24-27, wherein the disorder is hereditary angioedema (HAE).

29. The composition of claim 28, wherein the disorder is type I HAE or type II HAE.

30. The composition of any one of claims 24-29, wherein the at least one protein is a kallikrein-kinin system protein selected from the group consisting of kallikrein-13 (KLK13), kallikrein-14 (KLK14), 2-chain high molecular weight kininogen (KNG1), and kininostatin.

31. The composition of any one of claims 24-30, wherein the at least one protein is a blood coagulation protein selected from the group consisting of alpha-2-macroglobulin (A2M), complement C1q and tumor necrosis factor-related protein 9A (C1QTNF9), thrombin, and plasma serine protease inhibitor (SERPINA5).

32. The composition of any one of claims 24-31, wherein the at least one protein is a cell adhesion protein selected from the group consisting of cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), and protocadherin gamma-C3 (PCDHGC3).

33. The composition of any one of claims 24-32, wherein the at least one protein is a proteolysis-related protein selected from the group consisting of proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), and E3 ubiquitin-protein ligase ZNRF3 (ZNRF3).

34. The composition of any one of claims 24-33, wherein the at least one protein is a complement activation protein selected from the group consisting of complement C1q and tumor necrosis factor-related protein 9A (C1QTNF9).

35. The composition of any one of claims 24-34, wherein the at least one protein is selected from the group consisting of thrombin (F2), tissue kallikrein 14 (KLK4), tissue kallikrein 13 (KLK13), Inter-α-trypsin inhibitor heavy chain H4 (ITIH4), α-macroglobulin, Apolipoprotein B (APOB), interleukin-21 (IL-21), complement component C3 (C3), kininogen (KNG1), Protein arginine N-methyltransferase 1 (PRMT1), and Complement C1q and tumor necrosis factor-related protein 9A (C1QTNF9).

36. The composition of any one of claims 24-35, wherein the level of the biomarker set is measured by enzyme-linked immunosorbent assay (ELISA), an immunoblotting assay, or a lateral flow assay.

37. The composition of any one of claims 24-36, wherein the subject is a human patient.

38. A method for evaluating a treatment in a subject, comprising
measuring the level of a biomarker set, which comprises at least one protein selected from Table 1, in biological samples obtained from the subject before and after the treatment or during the course of the treatment, and
evaluating effectiveness of the treatment based on the level of the biomarker set, wherein a deviation level of the biomarker set after the treatment or over the course of the treatment as compared to before the treatment, indicates that the treatment is effective on the subject.

39. The method of claim 38, wherein the treatment comprises administration of a plasma kallikrein (pKal) inhibitor to the subject.

40. The method of claim 39, wherein the pKal inhibitor is lanadelumab.

41. The method of any one of claims 38-40, wherein the biomarker set consists of 2-10 proteins selected from Table 1.

42. The method of any one of claims 38-41, wherein the biological samples are serum samples or a plasma samples.

43. The method of any one of claims 38-42, wherein the at least one protein is a kallikrein-kinin system protein selected from the group consisting of kallikrein-13 (KLK13), kallikrein-14 (KLK14), 2-chain high molecular weight kininogen (KNG1), and kininostatin.

44. The method of any one of claims 38-43, wherein the at least one protein is a blood coagulation protein selected from the group consisting of alpha-2-macroglobulin (A2M), complement C1q and tumor necrosis factor-related protein 9A (C1QTNF9), thrombin, and plasma serine protease inhibitor (SERPINA5).

45. The method of any one of claims 38-44, wherein the at least one protein is a cell adhesion protein selected from the group consisting of cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), and protocadherin gamma-C3 (PCDHGC3).

46. The method of any one of claims 38-45, wherein the at least one protein is a proteolysis-related protein selected from the group consisting of proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), and E3 ubiquitin-protein ligase ZNRF3 (ZNRF3).

47. The method of any one of claims 38-46, wherein the at least one protein is a complement activation protein selected from the group consisting of complement C1q and tumor necrosis factor-related protein 9A (C1QTNF9).

48. The method of any one of claims 38-47, wherein the at least one protein is selected from the group consisting of thrombin (F2), tissue kallikrein 14 (KLK4), tissue kallikrein 13 (KLK13), Inter-α-trypsin inhibitor heavy chain H4 (ITIH4), α-macroglobulin, Apolipoprotein B (APOB), interleukin-21 (IL-21), complement component C3 (C3), kininogen (KNG1), Protein arginine N-methyltransferase 1 (PRMT1), and Complement C1q and tumor necrosis factor-related protein 9A (C1QTNF9).

49. The method of any one of claims 38-48, wherein the biological samples are obtained in evacuated blood collection tubes, which comprise one or more protease inhibitors.

50. The method of any one of claims 38-49, wherein the level of the biomarker set is measured by enzyme-linked immunosorbent assay (ELISA), an immunoblotting assay, or a lateral flow assay.

51. The method of any one of claims 38-50, wherein the subject is a human patient.

52. The method of any one of claims 38-51, wherein the subject has, is suspected of having, or is at risk for a disease associated with the contact activation system.

53. The method of claim 52, wherein the disease associated with the contact activation system is hereditary angioedema (HAE).

54. The method of claim 53, wherein the HAE is type I HAE or type II HAE.

55. The method of claim 54, wherein the disease associated with the contact activation system is not HAE.

56. A method for analyzing a sample, the method comprising:
(i) providing a set of biological samples obtained from a subject having, suspected of having, or being at risk for a disease associated with the contact activation system; and
(ii) measuring the level of a biomarker set, which comprises at least one protein selected from Table 1, wherein if the biomarker set consists of one protein, said protein is not high molecular weight kininogen (KNG1), alpha-2-macroglobulin (A2M), complement C1q and tumor necrosis factor-related protein 9A (C1QTNF9), thrombin, or interleukin-21 (IL-21).

57. The method of claim 56, wherein the biomarker set consists of 2-10 proteins selected from Table 1.

58. The method of claim 56 or 57, wherein the biological samples are serum samples or plasma samples.

59. The method of any one of claims 56-58, wherein the disease associated with the contact activation system is hereditary angioedema (HAE).

60. The method of claim 59, wherein the HAE is type I HAE or type II HAE.

61. The method of any one of claims 56-60, wherein the at least one protein is a kallikrein-kinin system protein selected from the group consisting of kallikrein-13 (KLK13), kallikrein-14 (KLK14), 2-chain high molecular weight kininogen (KNG1), and kininostatin.

62. The method of any one of claims 56-61, wherein the at least one protein is a blood coagulation protein selected from the group consisting of alpha-2-macroglobulin (A2M), complement C1q and tumor necrosis factor-related protein 9A (C1QTNF9), thrombin, and plasma serine protease inhibitor (SERPINA5).

63. The method of any one of claims 56-62, wherein the at least one protein is a cell adhesion protein selected from the group consisting of cadherin-1 (CDH-1), cadherin-15:cytoplasmic domain (CDH15), ephrin type-A receptor 2 (EPHA2), Multimerin-2 (MMRN2), olfactomedin-like protein 3 (OLFML3), and protocadherin gamma-C3 (PCDHGC3).

64. The method of any one of claims 56-63, wherein the at least one protein is a proteolysis-related protein selected from the group consisting of proteasome subunit beta type-6 (PSMB6), ubiquitin-conjugating enzyme E2 R2 (UBE2R2), ubiquitin-protein ligase E3A (UBE3A), ubiquitin conjugation factor E4 A (UBE4A), and E3 ubiquitin-protein ligase ZNRF3 (ZNRF3).

65. The method of any one of claims 56-64, wherein the at least one protein is a complement activation protein selected from the group consisting of complement C1q and tumor necrosis factor-related protein 9A (C1QTNF9).

66. The method of any one of claims 56-65, wherein the at least one protein is selected from the group consisting of thrombin (F2), tissue kallikrein 14 (KLK4), tissue kallikrein 13 (KLK13), Inter-α-trypsin inhibitor heavy chain H4 (ITIH4), α-macroglobulin, Apolipoprotein B (APOB), interleukin-21 (IL-21), complement component C3 (C3), kininogen (KNG1), Protein arginine N-methyltransferase 1 (PRMT1), and Complement C1q and tumor necrosis factor-related protein 9A (C1QTNF9).

67. The method of any one of claims 56-66, wherein step (i) comprises collecting the biological sample into an evacuated blood collection tube, which comprises one or more protease inhibitors.

68. The method of any one of claims 56-67, wherein step (ii) is performed using an enzyme-linked immunosorbent assay (ELISA), an immunoblotting assay, or a lateral flow assay.

69. The method of any one of claims 56-68, wherein the subject is a human patient.

70. The method of any one of claims 56-69, further comprising identifying the subject as a patient having the disease associated with the contact activation system, if the level of the biomarker set of the subject deviates from the level of the same biomarker set of a control subject.

71. The method of claim 70, further comprising administering to the subject an effective amount of a therapeutic agent, if the subject is identified as having the disease.

72. The method of any one of claims 56-71, wherein the subject is a human patient who has been administered a treatment for the disease and the method further comprises assessing the efficacy of the treatment by comparing the level of the biomarker set measured in a biological sample obtained from the subject after treatment or during the course of treatment to the level of the same biomarker set measured in a biological sample obtained from the subject before treatment;
wherein if the level of the biomarker set in the sample obtained after treatment or during the course of treatment deviates from the level of the biomarker set in the sample obtained before treatment, then the treatment is determined to be effective.

73. The method of any one of claims 56-72, wherein the subject is a human patient who has been administered a treatment for the disease and the method further comprises assessing the efficacy of the treatment by comparing the level of the biomarker set measured in a biological sample obtained from the subject after treatment or during the course of treatment to the level of the same biomarker set measured in a control sample obtained from a healthy subject;
wherein if the level of the biomarker set in the sample obtained after the treatment or during the course of the treatment does not substantially deviate from the level of the biomarker set in the control sample, then the treatment is determined to be effective.

74. The method of claim 72 or 73, further comprising administering to the subject an effective amount of a therapeutic agent or an increased dosage of the therapeutic agent, if the treatment is determined to not be effective.

75. The method of claim 74, wherein therapeutic agent is a plasma kallikrein (pKal) inhibitor, a bradykinin 2 receptor (B2R) inhibitor, and/or a C1 esterase inhibitor.

76. The method of claim 75, wherein the pKal inhibitor is an anti-pKal antibody or an inhibitory peptide.

77. The method of claim 76, wherein the pKal inhibitor is lanadelumab or ecallantide.

78. The method of claim 75, wherein the B2R inhibitor is an inhibitory peptide.

79. The method of claim 76, wherein the inhibitory peptide is icatibant.

80. The method of claim 75, wherein the C1 esterase inhibitor is a human plasma-derived C1 esterase inhibitor.

81. A kit for analyzing a sample of a subject having, suspected of having, or at risk for a disease associated with the contact system, the kit comprising:
(i) a first binding agent specific to a first protein biomarker selected from Table 1; and
(ii) a second binding agent specific to a second protein biomarker selected from Table 1;
wherein the first protein biomarker and the second protein biomarker are different and are not both selected from the group consisting of 2-chain high molecular weight kininogen (KNG1), alpha-2-macroglobulin (A2M), complement C1q and tumor necrosis factor-related protein 9A (C1QTNF9), thrombin, and interleukin-21 (IL-21).

82. The kit of claim 81, further comprising a first detection agent that binds to the first binding agent and a second detection agent that binds the second binding agent.

83. The kit of claim 81 or 82, wherein the first binding agent is an antibody specific to the first protein biomarker, and/or the second binding agent is an antibody specific to the second protein biomarker.

84. The kit of claim 81 or 82, wherein the first binding agent is an aptamer specific to the first protein biomarker; and/or the second binding agent is an aptamer specific to the second protein biomarker.

85. The kit of any one of claims 81-84, wherein the first binding agent and the second binding agent are immobilized on a support member.


SEQUENCE LISTING (13 sequences)

SEQ ID NO: 1 - protein, 304 aa, Homo sapiens
MIYTMKKVHALWASVCLLLNLAPAPLNADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 2 - protein, 58 aa, Bos taurus [sequence omitted]

SEQ ID NO: 3 - protein, 53 aa, synthetic construct
EAMHSFCAFKADGPCRAHPRWFNIFTRQCEFIYGCEGNQNRFESLECKMCTRD

SEQ ID NO: 4 - protein, 122 aa, synthetic construct
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSS

SEQ ID NO: 5 - protein, 105 aa, synthetic construct
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEI

SEQ ID NO: 6 - protein, 469 aa, synthetic construct
MGWSCILFLVATATGAHSEVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 231 aa, synthetic construct
MGWSCILFLVATATGAHSDIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 5 aa, synthetic construct
HYIMM

SEQ ID NO: 9 - protein, 17 aa, synthetic construct
GIYSSGGITVYADSVKG

SEQ ID NO: 10 - protein, 13 aa, synthetic construct
RRIGVPRRDEFDI

SEQ ID NO: 11 - protein, 11 aa, synthetic construct
RASQSISSWLA

SEQ ID NO: 12 - protein, 7 aa, synthetic construct
KASTLES

SEQ ID NO: 13 - protein, 8 aa, synthetic construct
QQYNTYWT